(19) United States
(12) Patent Application Publication
(10) Pub. No.: US 2023/0049979 A1
(43) Pub. Date: Feb. 16, 2023

(54) MACHINE LEARNING PREDICTION OF THERAPY RESPONSE

(71) Applicant: ONCOHOST LTD., Binyamina (IL)

(72) Inventors: Eran ISSLER, Zichron Yaakov (IL); Michal HAREL, Kfar Saba (IL); Coren LAHAV, Tel Aviv (IL); Eyal JACOB, Haifa (IL); Itamar SELA, Ramat-Gan (IL)

(21) Appl. No.: 17/797,245

(22) PCT Filed: Feb. 7, 2021
(86) PCT No.: PCT/IL2021/050147

§ 371 Date: Aug. 3, 2022

Related U.S. Application Data
(60) Provisional application No. 62/971,065, filed on Feb. 6, 2020.
(60) Provisional application No. 63/022,736, filed on May 11, 2020.
(60) Provisional application No. 63/089,304, filed on Oct. 8, 2020.

(51) Int. Cl. G16H 50/20; G16B 25/10; G16H 20/00

(52) U.S. Cl. CPC G16H 50/20; G16B 25/10; G16H 20/00

(57) ABSTRACT

A method comprising receiving, for each of a plurality of subjects having a specified type of disease and receiving a specified therapy for treating the disease, a first biological signature obtained pre-treatment and a second biological signature obtained on-treatment; calculating, for each of the plurality of subjects, a set of values representing a ratio between the first and second biological signatures associated with the respective subject; at a training stage, training a machine learning model on a training set comprising: (i) the calculated sets of values, and (ii) labels associated with an outcome of the specified therapy in each of the subjects; to generate a classifier suitable for predicting a response in a target patient to said specified therapy.

### CROSS REFERENCE TO RELATED APPLICATIONS

This application claims the benefit of priority under 35 U.S.C. § 119(e) of U.S. Provisional Application No. 62/971,065, filed on Feb. 6, 2020, 63/022,736, filed on May 11, 2020 and 63/089,304, filed on Oct. 8, 2020. The contents of the above applications are all incorporated by reference as if fully set forth herein in their entirety.

### FIELD OF THE INVENTION

The present invention relates to the field of machine learning.

### BACKGROUND

One of the major complications in various diseases, including but not limited to, oncology is resistance to therapy. Many studies have focused on the involvement of mutations and epigenetic changes in tumor cells in conferring drug resistance. However, in recent years, studies have indicated the contribution of the tumor microenvironment to therapy resistance, and that in response to almost any type of anti-cancer therapy, the patient (i.e., the host) may generate pro-tumorigenic and pro-metastatic processes that may counteract treatment effect.

The host-response to cancer treatment is relatively newly described phenomenon that has made a paradigm shift in understanding cancer progression and resistance to therapy, and is suggested in the present invention to be used for the early identification of non-responsive patients, and as a discovery tool for targets for medical intervention (e.g., selective inhibitors of key factors that can be co-administered with standard of care to improve treatment outcome in non-responding patients).

Therefore, there is a considerable need to identify biomarkers that can predict response to therapy.

The foregoing examples of the related art and limitations related therewith are intended to be illustrative and not exclusive. Other limitations of the related art will become apparent to those of skill in the art upon a reading of the specification and a study of the figures.

### SUMMARY OF INVENTION

The following embodiments and aspects thereof are described and illustrated in conjunction with systems, tools and methods which are meant to be exemplary and illustrative, not limiting in scope.

There is provided, in an embodiment, a system comprising at least one hardware processor; and a non-transitory computer-readable storage medium having stored thereon program instructions, the program instructions executable by the at least one hardware processor to: receive, for each of a plurality of subjects having a specified type of disease and receiving a specified therapy for treating the disease, (a) a first biological signature associated with a biological sample collected at a first time point relative to the specified therapy, and (b) a second biological signature associated with a biological sample collected at a second time point relative to the specified therapy, calculate, for each of the plurality of subjects, a set of values representing a relation between the first and second biological signatures associated with the respective subject, and at a training stage, train a machine learning model on a training set comprising: (i) the calculated sets of values, and (ii) labels associated with an outcome of the specified therapy in each of the subjects, to generate a classifier suitable for predicting a response in a target patient to said specified therapy.

There is also provided, in an embodiment, a method comprising: receiving, for each of a plurality of subjects having a specified type of disease and receiving a specified therapy for treating the disease, (a) a first biological signature associated with a biological sample collected at a first time point relative to the specified therapy, and (b) a second biological signature associated with a biological sample collected at a second time point relative to the specified therapy; calculating, for each of the plurality of subjects, a set of values representing a relation between the first and second biological signatures associated with the respective subject; and at a training stage, training a machine learning model on a training set comprising (i) the calculated sets of values, and (ii) labels associated with an outcome of the specified therapy in each of the subjects; thereby generate a classifier suitable for predicting a response in said target patient to said specified therapy.

There is further provided, in an embodiment, a computer program product comprising a non-transitory computer-readable storage medium having program instructions embodied therewith, the program instructions executable by at least one hardware processor to: receive, for each of a plurality of subjects having a specified type of disease and receiving a specified therapy for treating the disease, (a) a first biological signature associated with a biological sample collected at a first time point relative to the specified therapy, and (b) a second biological signature associated with a biological sample collected at a second time point relative to the specified therapy, calculate, for each of the plurality of subjects, a set of values representing a relation between the first and second biological signatures associated with the respective subject, and at a training stage, train a machine learning model on a training set comprising: (i) the calculated sets of values, and (ii) labels associated with an outcome of the specified therapy in each of the subjects, to generate a classifier suitable for predicting a response in said target patient to said specified therapy.

In some embodiments, the first and second biological signatures are each one of: a DNA profile, an RNA profile, a protein profile, a metabolomics profile, microbiome profile, a transcriptomics profile, a genomics profile, an epigenomics profile, a cellular profile, a post-translational modification-based profile, single-cell based analysis, and a regulatory RNA profile.

In some embodiments, the first and second biological signatures are each protein expression profiles, and the sets of values each comprise, with respect to each protein in the protein expression profiles, a relation between the levels of expression of the protein in the first and second biological signatures.

In some embodiments, the protein expression profile comprises expression values for at least two proteins.

In some embodiments, the method further comprises performing, and the program instructions are further executable to perform, a dimensionality reduction stage with respect to the sets of values, to reduce the number of variables in at least one of the sets of values.

In some embodiments, the dimensionality reduction stage identifies a subset of principal proteins in each of the sets of values. In other embodiments, the dimensionality reduction generates a new feature that can be predictive for response.

In some embodiments, the dimensionality reduction involves regarding all or some feature values as vector components and calculating its norm.

In some embodiments, the training set comprises only the subset of principal proteins in each of the sets of values.

In some embodiments, the sets of values are labeled with the labels.

In some embodiments, each of the biological samples is one of: blood plasma, whole blood, blood serum, cerebrospinal fluid (CSF), and peripheral blood mononuclear cells (PBMCs).

In some embodiments, the specified type of disease is a specified type of cancer. In some embodiments, the cancer is selected from melanoma, non-small cell lung cancer (NSCLC), small cell lung cancer (SCLC), head and neck cancer and urogenital cancer.

In some embodiments, the training set further comprises, with respect to at least some of the subjects, labels associated with clinical data.

In some embodiments, the predicting is expressed as one of: a binary value, continuous value, and a set of discrete values.

In some embodiments, the predicting comprises an indication of secondary effects in the target subject.

In some embodiments, the method further comprises at an inference stage, applying said classifier to a target set of said values associated with a target subject, thereby predicting a response in said target subject to said specified therapy.

In some embodiments, the method further comprises determining, and the program instructions are further executable to determine, based, at least in part, on the predicting, at least one of: continuing the specified therapy in the target subject, adjusting the specified therapy in the target subject, discontinuing the specified therapy in the target subject, and administering a different therapy to the target subject.

In some embodiments, the specified therapy is an immunotherapy. In some embodiments, the specified therapy is a combination of immunotherapy and chemotherapy. In some embodiments, the specified therapy is a combination of immunotherapy and targeted therapy. In some embodiments, the specified therapy is a combination of more than one type of immunotherapy. In some embodiments, the immunotherapy is selected from anti-PD-1/PD-L1 therapy, anti-CTLA-4 therapy, and both.

In some embodiments of the system, computer program product, and method provided herein, adjusting the specified therapy or administering a different therapy to said target subject is determined by a method comprising: (i) determining differentially expressed proteins (DEPs) between responders and non-responders; (ii) determining, in the sample obtained from said subject, one or more resistance associated proteins (RAPs) selected from the determined DEPs; and (iii) selecting a therapy suitable for balancing the level of the one or more RAPs in said subject.

In some embodiments, determining the one or more RAPs is by providing a probabilistic measurement of the distance of the DEP expression level from a defined group of samples.

In some embodiments, determining the one or more RAPs in a subject is by determining the expression distribution of each DEP in each of the responder and non-responder groups, fitting a probability density function for each group, and calculating for each subject and based on the DEP expression of said subject, the probability of the DEP to be associated with one of the response groups. In specific embodiments, determining the one or more RAPs in a subject is by determining the probability of each DEP to be associated with the responder's distribution. In other embodiments, determining the one or more RAPs in a subject is by determining the probability of each DEP to be associated with the non-responder distribution.

In some embodiments, the therapy for balancing the level of the one or more RAPs in said subject is selected from a list of approved drugs or an investigational drug.

In addition to the exemplary aspects and embodiments described above, further aspects and embodiments will become apparent by reference to the figures and by study of the following detailed description.

### BRIEF DESCRIPTION OF THE FIGURES

FIG. 1 is a flowchart of the functional steps in a method for training a machine learning model to predict patient response to therapy, according to some embodiments of the present disclosure;

FIG. 2 is a schematic illustration of the process steps of FIG. 1, according to some embodiments of the present disclosure;

FIG. 3 is a non-limiting schematic illustration of a quality control process based on a limit of detection (LOD) threshold, according to some embodiments of the present disclosure;

FIGS. 4, 5A-5D, 6A-6C, and 7A-7C illustrate experimental results, according to some embodiments of the present disclosure (TP—true positive; FN—false negative; TN—true negative; FP—false positive, PPV—positive predictive value, NPV—negative predictive value);

FIG. 8 is a flowchart of the 3 filters for analysis of personalized potential targets for intervention, according to some embodiments of the present disclosure. Solid and dashed lines indicate a positive and a negative answer to the examined question, respectively. On the left, the analysis/data processing steps are indicated, followed by the applied filters. The clinical filter appears in the flowchart three times. F1 designates the cohort-based statistical filter; F2 designated the personalized filter; F3 designates the clinical filter;

FIG. 9 is a non-limiting example for the RAP score calculation. The example in this figure shows the protein distributions of an exemplary protein (“Protein A”) of the R (light blue) and NR (orange) in the entire cohort (n=52). A patient with Protein A expression level of 0.3, marked in a dashed line, has a P(NR)/P(R) ratio of 8, as calculated based on the area above 0.3 in the NR and in R distributions (the areas are marked by filled color). Following log 2 transformation, the RAP score of this protein in this specific patient is 3;

FIGS. 10A-10B depict a non-limiting example of RAP score directionality. The selection of areas for the RAP score calculation depends on the relative location of the R and NR distributions. A. If the median of the NR distribution of the given differentially expressed proteins (DEP) is higher than the median of the R distribution, the areas for equation 1 are calculated based on the right tail. B. If the median of the R distribution of the given DEP is higher than the median of the NR distribution, the areas for equation 1 are based on the left tail; and

FIGS. 11A-11C show that the RAP score distribution may depend on the difference between R and NR distributions. The RAP score is indicated above the plot. A. The R and NR distributions of Protein A expression levels or T1/T0. B. The R and NR distributions of Protein B expression levels or T1/T0. C. The distribution of Protein B RAP scores among NR patients.

FIG. 11D shows that the RAP score can be further used to identify groups of non-responders that share similar RAP profiles.

FIGS. 12A-12B show a simulation of RAP perturbation. (FIG. 12A) A predictive signature that is based on the list of RAPs of the entire cohort is generated. (FIG. 12B) For a given patient, a specific RAP (or RAPs) is perturbed. Next, the baseline response probability is compared to the perturbed response probability.

FIGS. 13A-13B demonstrate classifier training (FIG. 13A) and validation (FIG. 13B) based on the presented invention to predict response to treatment in psoriasis patients. FIG. 13A: (Left) SVM yielded an AUC of 0.77. (Right) Accuracy=0.7286, sensitivity=0.75, specificity=0.6818, PPV=0.8372 and NPV=0.5556. FIG. 13B: (Left) SVM yielded an AUC of 0.751. (Right) Accuracy=0.6714, sensitivity=0.6458, specificity=0.7273, PPV=0.8378 and NPV=0.4848. TP—true positive; FN—false negative; TN—true negative; FP—false positive, PPV—positive predictive value, NPV—negative predictive value.

FIG. 14 demonstrates differential network analysis. Networks of correlation data were constructed separately for each group. From these group-specific maps, a differential map can be generated to identify the proteins that are differentially correlated in each group.

FIG. 15 shows an example for a differential network between responders and non-responders based on the NSCLC dataset.

FIGS. 16A-16B show prediction based on protein co-changes. As a non-limiting example, two proteins showing differentially-correlated fold-change values between responders and non-responders were inspected. (16A) Correlation between responders and non-responders was positive (R=0.37). The dashed line shows a linear fit to the responder values. (16B) The residuals (i.e., distances of each point from the linear fit in A) of two protein pairs was calculated and used as input for an SVM classifier. The resulting predictor achieved an ROC AUC of 0.77.

FIG. 17. Preliminary results for response prediction using the naïve predictor in n=67 NSCLC patients. The response quality is quantified by the Area Under the Curve (AUC) of the Receiver-Operator Curve (ROC), for training set composed of n=37 responders and independent validation set containing n=15 responders and n=15 non-responders. The AUC was calculated for 1000 different sets of training and validation sets, where the n=37 responders training set were randomly sampled from the total n=52 responders in the dataset. The resulting 1000 AUC values are shown in a histogram, where the median value is shown in solid gray vertical line. The mean value of a random classifier AUC=½ is indicated by a vertical dashed line. For comparison, the AUC distribution of random classifiers for n=15 responders and n=15 non-responders are shown in white shading.

### DETAILED DESCRIPTION

Disclosed are a system, method, and computer program product which provide for a machine learning model configured to predict patient response to therapy. Further disclosed are a system, method, and computer program product which indicate a suitable alternative or accompanying therapy to improve the therapeutic outcome in a patient.

In some embodiments, the present disclosure provides for training a machine learning model using a training dataset comprising a biological profile (e.g., protein expression profile) of biological samples or biological signatures obtained from a plurality of subjects, e.g., a cohort or predefined population, having a specified type of disease and receiving a specified type of treatment (e.g., a therapy associated with the specified type of disease).

In certain embodiments, the cohort or predefined population of subjects is based on, or determined according to, any one of: disease type, disease stage, disease therapy, treatment history, clinical profile, and any combination thereof.

In some embodiments, a trained machine learning model of the present disclosure may provide for predicting a response of a target patient, diagnosed with the specified disease, to the associated specified treatment or therapy. In some embodiments, a machine learning model of the present disclosure may be trained on data from a cohort or a predefined population of subjects having a specified disease or type of disease, wherein a biological sample is obtained from a cohort participant at at-least one time point relative to the treatment, e.g., at T₀ (e.g., pre-treatment) or T₁ (e.g., during-, on- or post-treatment).

In some embodiments, the present disclosure further provides for a process for the identification and characterization of host response to a specified therapy. In some embodiments, the present disclosure is based, at least in part, on identifying one or more biological signatures that differ at two time points relative to a specified treatment, in order to predict therapy effectiveness and outcome.

In some embodiments, a machine learning model of the present disclosure may be trained on data from a cohort or a predefined population of subjects having a specified disease or type of disease, wherein at least two biological samples are obtained from each cohort participant at two time points relative to the treatment, e.g., at T₀ (e.g., pre-treatment) and T₁ (e.g., during-, on- or post-treatment). In some embodiments, the biological samples are profiled to extract a biological signature, e.g., a protein expression profile.

Accordingly, in some embodiments, the present disclosure provides (i) a computational approach for training a machine learning model to predict a response in patients, as well as (ii) methods for selecting key proteins whose targeting may improve therapy efficacy and/or response to therapy.

The present disclosure will discuss aspects of the present invention associated with predicting response, e.g., host response, in cancer patients. The term “host response” as used herein refers to a set of patient-driven factors that may limit or counteract the effectiveness of one or more cancer treatment or therapy modalities applied to the patient. However, the present method may be equally effective in predicting treatment and/or therapy response in the context of other diseases or disorders. Further, the present method may be effective for patient population enrichment such as for use in clinical trials. Further, the present method may be effective to identify novel combinations of therapies suitable for treating a subject.

In some embodiments, biological samples may be obtained from each subject in a cohort of patients, or from at least some of the subjects, at specified times before, during, and/or after the conclusion of, the course of therapy. In some embodiments, the biological samples may be obtained from each subject, or from at least some of the subjects, at specified one or more stages and/or points and/or steps before, during, and/or after the conclusion of, the course of therapy, e.g., pre-therapy, on-therapy, and/or post-therapy.

In some embodiments, biological signatures (e.g., protein expression profiles) may be obtained from each of the biological samples. In some embodiments, a set of biological signatures may comprise statistically-tested biological signatures obtained at multiple times (e.g., T₀ and T₁) from a cohort of subjects undergoing a specified therapy. In some embodiments, a preprocessing stage may take place to preprocess the biological signature data. In some embodiments, the preprocessing stage may comprise at least one of data cleaning and normalizing, feature selection, feature extraction, dimensionality reduction, and/or any other suitable preprocessing method or technique.

In some embodiments, the paired biological signatures associated with each subject may be analyzed to determine a differential expression within each pair, e.g., values associated with differentially expressed factors (e.g., proteins) in the paired biological signatures. In some embodiments, this analysis provides for a difference in the relation between at least some of the proteins in each signature. In some embodiments, this analysis provides for a set of values representing a difference in expression of at least some factors (e.g., proteins) in the paired biological signatures of each of, or at least some of, the subjects. In some embodiments, the set of values representing a relation between at least some factors (e.g., proteins) in the paired biological signatures may be based on one or more mathematical equations, such as multiplication of the expression values or a difference in the relation between the expression values. In some embodiments, the ratio is between biological signatures at T₀ and T₁. In some embodiments, the ratio is between biological signatures at T₁ and T₀. As used herein, the term “paired biological signatures”, “pairs of biological signatures”, and variations thereof, refers to biological signatures obtained from multiple (i.e., two or more) biological samples received at multiple time points relative to the specified therapy. As such the analysis may compare the multiple biological signatures and provide a pattern of the signature over time. In some embodiments, monitoring progress of a diseased state of a patient may require multiple sampling of biological signatures from the patient.

Accordingly, in some embodiments, a training dataset for a machine learning model of the present disclosure may comprise a plurality of sets of values associated with a difference and/or ratio in expression of at least some proteins in associated pairs of biological signatures of each of, or at least some of, a cohort of subjects having each a specified type of disease and receiving each a specified type of treatment and/or therapy associated with the specified type of disease.

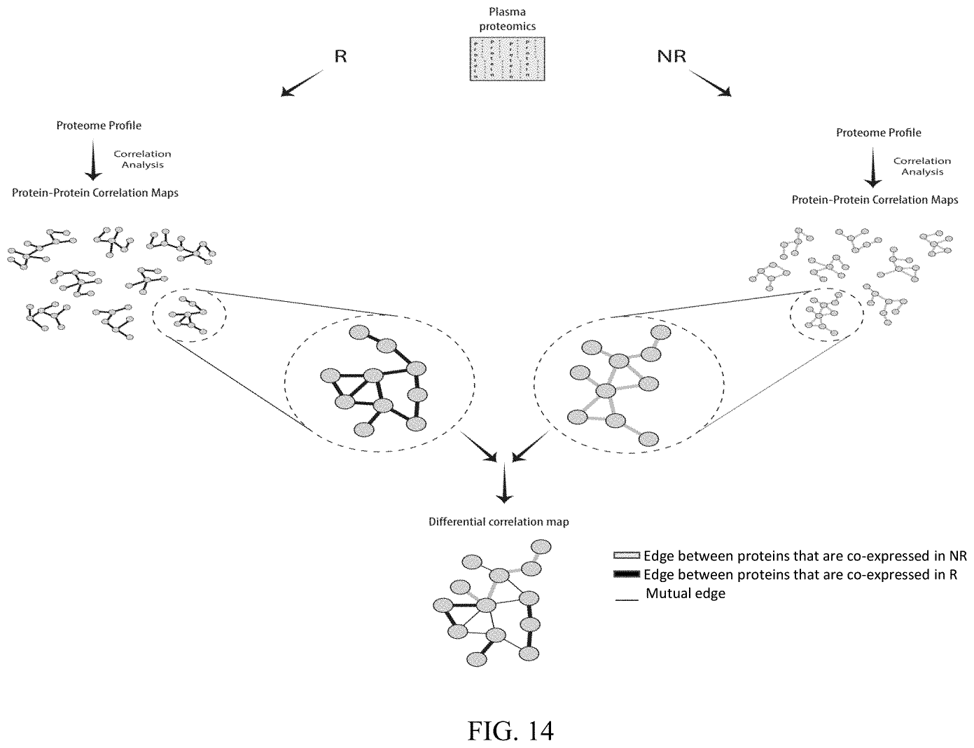

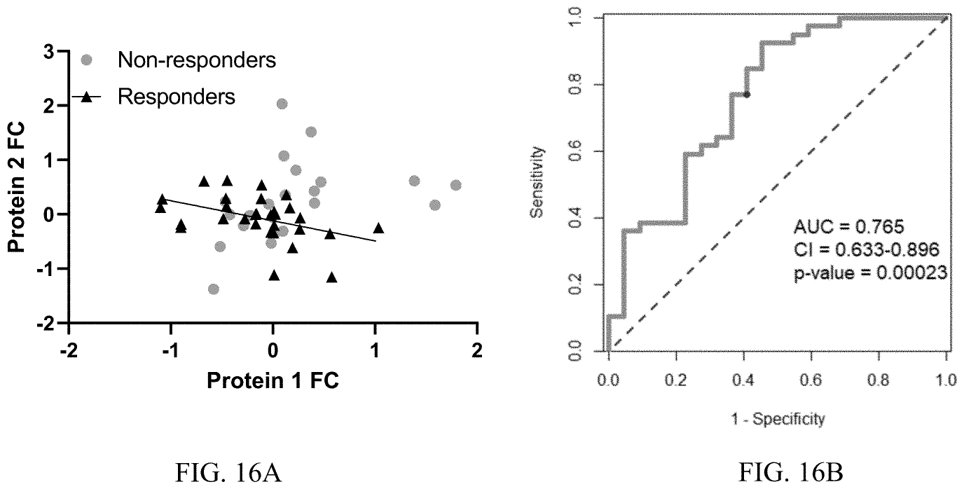

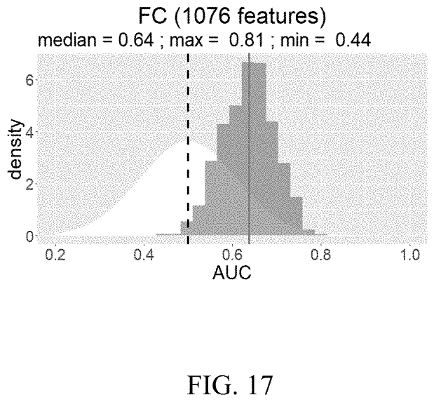

In some embodiments, the paired biological signatures may be correlated using the same factor (e.g., the same protein). In some embodiments, the paired biological signatures may be correlated under a plurality of factors (e.g., various proteins) which define a network of factors (e.g., a protein network). As demonstrated hereinbelow (FIGS. 14-16), differential correlations of proteins can also provide a tool for feature engineering useful for prediction of response of a subject to a specified therapy. As a non-limiting example, a protein network can be defined for each biological signature and a calculation is performed to define the overall behavior of each cohort (e.g., a calculation of the distance from the trendline of the correlation, as demonstrated under FIG. 16A).

In some embodiments, a training dataset for machine learning model of the present disclosure may comprise a plurality of sets of values associated with a difference (e.g., ratio) in expression of at least some factors (e.g., proteins) in associated biological signatures of each of, or at least some of, a cohort of subjects having a specified type of disease and receiving a specified type of treatment and/or therapy associated with the specified type of disease, wherein at least some of the sets of values may be annotated with category labels denoting a response and/or outcome of the treatment in the respective subject.

In some embodiments, a training dataset for a machine learning model of the present disclosure comprises, e.g., a plurality of sets of values associated with a difference (e.g., ratio) in expression of at least some factors (e.g., proteins) in associated biological signatures of each of, or at least some of, a cohort of subjects having a specified type of disease and receiving a specified type of treatment and/or therapy associated with the specified type of disease, wherein at least some of the sets of values may be annotated with category labels denoting a response and/or outcome of the treatment in the respective subject, wherein the annotation may be binary, e.g., positive/negative, responsive/non-responsive, continuous, and/or expressed on any numeric scale, e.g., of 1-5 or complete response, partial response, overall response, duration of response, progression-free survival, adverse events, stable disease, or progressive disease, or the like. In some embodiments, additional and/or other annotation schemes may be employed and used for the training dataset. In some embodiments, the training dataset may be annotated with category labels denoting, e.g., patient demographic and/or clinical data.

In some embodiments, a trained machine learning model of the present disclosure may provide for predicting a response of a patient diagnosed with a specified disease to the associated specified treatment or therapy.

In some embodiments, a trained machine learning model of the present disclosure provides for predicting a response of a patient to the specified treatment or therapy as a binary value, e.g., ‘yes/no,’ ‘responsive/non-responsive,’ or ‘favorable/non-favorable response.’ In some embodiments, the prediction may be expressed by values indicating a response probability (e.g., at a scale of 1-100%). In some embodiments, the prediction may be expressed on a scale and/or be associated with a confidence parameter. Accordingly, in some embodiments, a machine learning model of the present disclosure may provide for predicting a response rate and/or success rate of a specified treatment in a patient, e.g., the likelihood of a favorable response of a patient to the specified treatment or therapy. For example, in some embodiments, the prediction may be expressed in discrete categories and/or on a scale comprising, e.g., ‘complete response,’ partial response,′ ‘stable disease,’ ‘progressive disease,’ ‘pseudo-progression’ and ‘hyper-progression disease.’ In some embodiments, the prediction may indicate adverse or any other secondary effects, e.g., side-effects based on the host response. In some embodiments, the prediction may indicate whether a response by a patient is associated with adverse or any other secondary effects. In some embodiments, the prediction may indicate the overall response of the patient to the specified treatment or therapy. In some embodiments, the prediction may indicate the progression-free survival rate following treatment of the patient with the specified treatment or therapy. In some embodiments, the prediction may indicate the duration of response rate of the patient. In some embodiments, additional and/or other scales and/or thresholds and/or response criteria may be used, e.g., a gradual scale of 1 (non-responsive) to 5 (responsive).

In some embodiments, the present disclosure may provide also for predicting adverse events associated with the specified treatment or therapy of a target patient. In some embodiments, the present disclosure may provide also for predicting metastasis, metastasis location and/or tumor burden in a target patient.

In some embodiments, the present disclosure may provide for predicting the overall response, duration of response and progression-free survival of a target patient treated with the specified treatment or therapy.

In the context of cancer, the term “therapy” refers to any method of treatment of a specified disease in a subject. In the context of cancer, the terms “therapy”, “anti-cancer therapy”, “cancer therapy modality”, “treatment modality”, “cancer treatment”, or “anti-cancer treatment”, as used herein, refer to any method of treatment of cancer in a cancer patient including radiotherapy; chemotherapy; targeted therapy, immunotherapy (immune checkpoint inhibitors, immune checkpoint modulators, adoptive-cell transfer therapy, oncolytic viruses therapy, treatment vaccines, immune system modulators and monoclonal antibodies), hormonal therapy, anti-angiogenic therapy and photodynamic therapy; thermotherapy and surgery or a combination thereof. In some embodiments, the cancer therapy is immunotherapy. In some embodiments, the immunotherapy comprises immune checkpoint modulation. In some embodiments, the immunotherapy comprises immune checkpoint inhibition. In some embodiments, inhibition comprises administering an immune checkpoint inhibitor. In some embodiments, the inhibitor is a blocking antibody. In some embodiments, the immunotherapy comprises immune checkpoint blockade. Immune checkpoint proteins are well known in the art and include, but are not limited to PD-1, PD-L1, PD-L2, CTLA-4 (Cytotoxic T-Lymphocyte-Associated protein 4); A2AR (Adenosine A2A receptor), also known as ADORA2A; B7-H3, also called CD276; B7-H4, also called VTCN1; B7-H5; LAG-3 (Lymphocyte Activation Gene-3); BTLA (B and T Lymphocyte Attenuator), also called C272; TIM-3 (T-cell Immunoglobulin domain and Mucin domain 3); IDO (Indoleamine 2,3-dioxygenase); TDO (Tryptophan 2,3-dioxygenase); KIR (Killer-cell Immunoglobulin-like Receptor); NOX2 (nicotinamide adenine dinucleotide phosphate NADPH oxidase isoform 2); SIGLEC7 (Sialic acid-binding immunoglobulin-type lectin 7), also called CD328; SIGLEC9 (Sialic acid-binding immunoglobulin-type lectin 9), also called CD329, TIGIT and VISTA (V-domain Ig suppressor of T cell activation). In some embodiments, the immunotherapy is anti-PD-1 therapy. In some embodiments, the immunotherapy is anti-PD-L1 therapy. In some embodiments, the immunotherapy is anti-PD-L1/PD-L2 therapy. In some embodiments, the immunotherapy is combined with another immunotherapy. In some embodiments, the immunotherapy is anti-PD-1 and/or anti-PD-L1 therapy. In some embodiments, the immunotherapy is anti-CTLA-4 therapy. In some embodiments, the immunotherapy is anti-PD-1 and anti-CTLA-4 therapy. In some embodiments, the immunotherapy is anti-PD-L1 and anti-CTLA-4 therapy. In some embodiments, the immunotherapy is combined with another treatment modality. In some embodiments, the treatment modality is another anticancer treatment. Examples of other anticancer treatments include but are not limited to chemotherapy, radiation, surgery, and targeted therapy. Any other anticancer treatment may be combined. In some embodiments, the immunotherapy is combined with chemotherapy. In some embodiments, the immunotherapy is combined with targeted therapy. In some embodiments, the immunotherapy is a combined with more than one type of an additional immunotherapy. In some embodiments, the immunotherapy is selected from anti-PD-1/PD-L1 therapy, anti-CTLA-4 therapy, and both.

In some embodiments, the additional treatment modality is a treatment against side effects of the immunotherapy. The side effects of anticancer therapeutics in general, and immunotherapy, are well known. Any such anti-side effect treatment may be employed, including, but not limited to steroids, folic acid and the like.

In certain embodiments, the terms “treatment” or “therapy” refer to one or more sessions of treatment of a patient. In specific embodiments, the term “pre-treatment” refers to a time point before a session of a specified treatment, and the term “on treatment” refers to a time point after the session of treatment and before the next session of treatment. In alternative specific embodiments, the term “on treatment” refers to a time point between the second- and third-sessions of treatment; between the third- and forth-sessions of treatment; between the fourth- and fifth-sessions of treatment; etc. In some embodiments, the term “post-treatment” refers to a time point after the completion of the treatment. In specific embodiments, the term “post-treatment” refers to a time point after progression was identified.

In specific embodiments, the term “pre-treatment” refers to a time point before the first session of a specified treatment, and the term “on-treatment” refers to a time point after the first session of treatment and before the second session of treatment.

In some embodiments, aspects of the invention further provide for monitoring the responsiveness of the patient to a therapy over time. In such embodiments, the analysis may provide for a difference in the relation between at least some of the proteins in each signature between two or more time points following treatment (e.g., T₂ and T₃ etc.). The difference in relation between T₁ and T₀ is presented in the application only for the purpose of illustration. Other differences or ratios are also applicable e.g., T₂/T₁, T₃/T₂, T₄/T₃, Tn+₁/Tn, Tn+x/Tn and the same, and T₂/T₀, T₃/T₀ T₄/T₀, Tn/T₀.

In some embodiments, paired T₀ and T₁ expression profiles, may correspond to before and after a specified one of the sessions of treatment, which may be the first, second, third, and/or another session of treatment. In such a case, a first expression profiles means obtaining data from biological samples collected from a subject prior to receiving a specified one of the sessions of treatment; and a second expression profile means obtaining data from biological samples collected from the subject after receiving the specified one of the sessions of treatment.

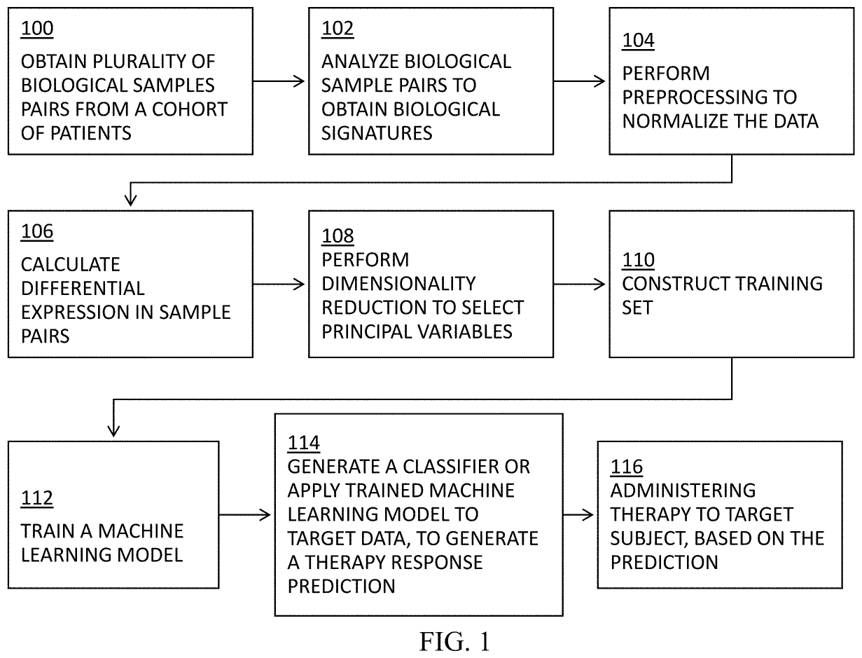

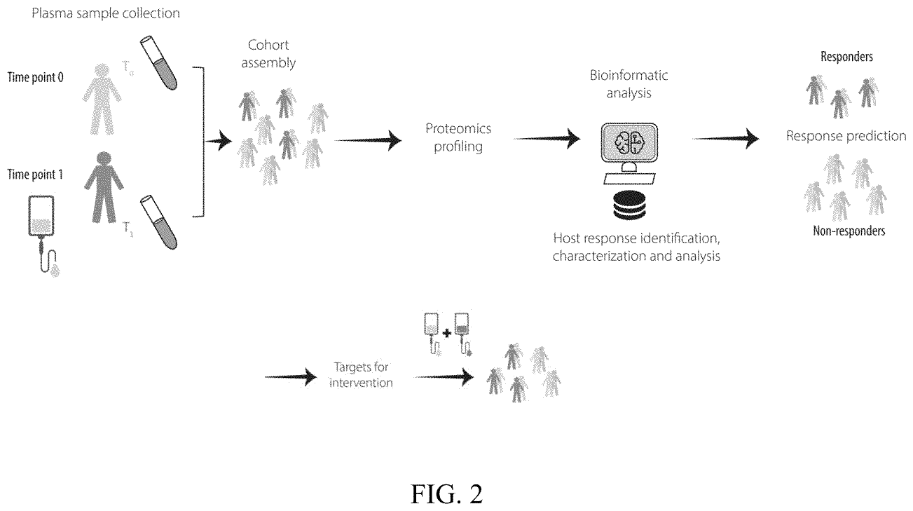

FIG. 1 is a flowchart of the functional steps in a method for training a machine learning model to predict patient response to therapy, according to some embodiments of the present disclosure. FIG. 2 is a schematic illustration of the process steps of FIG. 1.

In some embodiments, at step 100, a plurality of biological samples may be received from a cohort of subjects, e.g., a predefined population of patients having a specified type of disease. In some embodiments, a cohort assembled for the purposes of the present disclosure may comprise a plurality of patients having the same and/or a similar and/or an associated disease and/or category of diseases and/or syndromes and/or conditions, and/or associated diseases, syndromes and/or conditions. In some embodiments, with respect to at least some of the patients in the cohort, the specified disease and/or conditions may be at different stages and/or be combined with co-morbidities and/or diseases. In some embodiments, a specified disease of the present disclosure may be expressed in terms of broad categories (e.g., ‘cancer’), sub-types (e.g., melanoma), and/or sub-categories (e.g., a specified type of melanoma).

In some embodiments, the disease is a proliferative disorder. In some embodiments, the disease is a disease characterized, by increased proliferation, decreased apoptosis, or both. In some embodiments, the disease is cancer. In some embodiments, the cancer is a solid cancer. In some embodiments, the cancer is a hematopoietic cancer. Types of cancer are well known in the art, and examples of classes of cancer include, but are not limited to a sarcoma, a melanoma, a blastoma, a carcinoma, a leukemia and a lymphoma. Types of cancer can also be classified by the tissue/cell type of origin and include for example, brain cancer, blood cancer, bone cancer, fat cancer, retinoblastoma, head and neck cancer, tongue cancer, nasopharyngeal cancer, pharyngeal cancer, throat cancer, esophageal cancer, stomach cancer, gastrointestinal cancer, intestinal cancer, lung cancer, colon cancer, colorectal cancer, liver cancer, pancreatic cancer, gallbladder cancer, penile cancer, thymus cancer, thyroid cancer, urogenital cancer, prostate cancer, kidney cancer, ovarian cancer, cervical cancer, testicular cancer, skin cancer, glioblastoma multiforme (GBM), and uterine cancer. In some embodiments, the cancer is skin cancer. In some embodiments, the cancer is lung cancer. In some embodiments, the cancer is melanoma. In some embodiments, the cancer is small cell lung cancer (SCLC) or non-small cell lung cancer (NSCLC). In some embodiments, the cancer is urogenital cancer. In some embodiments, the cancer is head and neck cancer. In some embodiments, the cancer is a solid tumor. In some embodiments, the cancer is a cancer treatable by immunotherapy.

In some embodiments, the disease is an autoimmune disease. In some embodiments, the autoimmune disease is psoriasis. In some embodiments, the disease is a genetic disease. In some embodiments, the disease is an infectious disease. In some embodiments, the disease is a bacterial, viral or fungal infection. In some embodiments, the disease is an inflammatory disease. In some embodiments, the disease is a respiratory disease. In some embodiments, the disease is degenerative disease. In some embodiments, the disease is a neurodegenerative disease. In some embodiments, the disease is a metabolic disease. In some embodiments, the disease is a cardiovascular disease. In some embodiments, the disease is a skeletal disease.

In some embodiments, biological samples may include any type of biological sample obtained from an individual, including body tissues, body fluids, body excretions, exhaled breath, or other sources. In some embodiments, the biological sample is a tumor. In some embodiments, the biological sample is a non-tumorigenic sample. Body fluids may be whole blood, blood plasma, blood serum, peripheral blood mononuclear cells (PBMCs), lymph, urine, saliva, semen, synovial fluid and spinal fluid, fresh or frozen. In certain embodiments of the method according to the invention, the biological sample(s) is blood plasma, whole blood, blood serum, cerebrospinal fluid (CSF), or PBMCs. In specific embodiments, the biological sample(s) is blood plasma. In alternative specific embodiments, the biological sample(s) is CSF. In some embodiments, the biological sample(s) is PBMCs sample. In some embodiments, the biological sample(s) is a blood sample.

In some embodiments, a cohort of the present disclosure comprises a group of subjects with similar phenotype and receiving a similar treatment. However, the cohort definition may vary according to the classification per cohort and biological common denominator of the participating subjects. In some embodiments, a cohort of the present disclosure may comprise patients of, e.g., different demographics (e.g., sex, age, ethnicity), clinical measurements, disease stage, disease history, disease treatment history, general medical history (e.g., including smoking history and drinking habits, background diseases) genetic information, physical parameters, and the like.

In some embodiments, patients in the cohort may undergo and/or receive different types of treatments, e.g., mono therapy, combined therapy, multi-stage or multi-session therapy, and/or multi-modality therapy.

In some embodiments, the biological samples may be obtained from each subject in the cohort, or from at least some of the subjects, at specified times before, during, and/or after the conclusion of, the course of therapy. In some embodiments, the biological samples may be obtained from each subject, or from at least some of the subjects, at specified one or more stages and/or points and/or steps before, during, and/or after the conclusion of, the course of therapy, e.g., pre-therapy, on-therapy, and/or post-therapy.

In some embodiments, with respect to at least some of the subjects, at least a pair of corresponding T₀ and T₁ biological samples may be collected at two or more different points during the course of the treatment, e.g.:
(i) pre-therapy, i.e., before the start of the course of therapy, and (ii) post-therapy, i.e., after the conclusion of the entire course of therapy;
(i) pre-therapy, i.e., before the start of the course of therapy, and (ii) on-therapy, i.e., at a specified point in time during the course of therapy;
in the case of a multi-stage or multi-session treatment, (i) pre-therapy, i.e., before the start of the course of therapy, and (ii) after the conclusion of a specified stage and/or session of the multi-stage or multi-session therapy; and/or
in the case of a multi-modality therapy, (i) pre-therapy, i.e., before the start of the course of therapy, and (ii) at a specified point and/or stage associated with one of the multiple treatment modalities.
in the case of a multi-modality therapy, (i) pre-therapy, i.e., before the start of the course of therapy for each treatment modality, and (ii) at a specified point and/or stage associated with one of the multiple treatment modalities.

In some embodiments, at step 102, each of, or at least some of, the biological samples may be analyzed to identify a plurality of biomarkers and/or to extract a biological signature. In some embodiments, the analysis obtains, e.g., a proteomic profile comprising protein expression for each of the samples. In some embodiments, the protein expressions so obtained may identify the proteins in each analyzed biological sample. In some embodiments, additional and/or other analyses may be performed with respect to the biological samples, to obtain, e.g., one or more profiles selected from: DNA profile; RNA profile; circulating DNA profile, single cell RNA sequencing; metabolomics; microbiome; transcriptome; genomics; epigenomics; cell profiling; single-cell based analysis; and MicroRNA. In some embodiments, the circulating DNA profile is circulating tumor DNA profile. In some embodiments, the circulating DNA profile is methylated circulating DNA profile.

In certain embodiments, the biological signature is selected from: a proteome profile; a DNA profile; an RNA profile; a metabolomics profile (e.g., glycomics, lipidomics); a microbiome profile; a genomics profile; an epigenomics profile; a cellular profile; a post-translational modification-based profile; a single-cell based analysis; and a regulatory RNA profile. In some embodiments, expression is protein expression. In some embodiments, expression is RNA expression. In some embodiments, the RNA is mRNA. In some embodiments, the RNA is regulatory RNA. In some embodiments, the regulatory RNA is microRNA. In some embodiments, the regulatory RNA is a long non-coding RNA. In some embodiments, the metabolomics profile is lipids profile. In some embodiments, the metabolomics profile is nucleic acids profile. In some embodiments, the metabolomics profile is sugars profile. In some embodiments, the metabolomics profile is vitamins profile. In some embodiments, the metabolomics profile is fatty acids profile. In some embodiments, the metabolomics profile is amino acids profile. In some embodiments, the metabolomics profile is phenolic compounds profiles. In some embodiments, the metabolomics profile is alkaloids profiles. In some embodiments, the protein expression is metabolic protein expression. In some embodiments, the protein expression is membranal protein expression. In some embodiments, the protein expression is secreted protein expression. In some embodiments, the protein expression is cellular protein expression. In some embodiments, the biological signature is a profile of the genome. In some embodiments, the biological signature is a mutational profile of the genome. In some embodiments, the biological signature is an epigenetic profile of the genome. In some embodiments, the biological signature is a methylome profile. In some embodiments, the epigenetic profile is a profile of post-translational modifications (PTMs). In some embodiments, the biological signature is PTM profile on proteins. PTMs are well known in the art and include, but are not limited to, methylation, acetylation, phosphorylation, glycosylation, sumoylation, and ubiquitination. In some embodiments, the biological signature is a circulating DNA profile. In some embodiments, the biological signature is circulating tumor-DNA profile. In some embodiments, the biological signature is methylated circulating tumor DNA profile. In some embodiments, the biological signature is amount of circulating tumor DNA profile. In some embodiments, the biological signature is genotyping of mutations in circulating tumor DNA profile. In some embodiments, the biological signature is an organismal profile. In some embodiments, the biological signature is a microbiome profile. In some embodiments, the biological signature is extracellular vesicles profile (either number or content). In some embodiments, the biological signature is microparticles profile (either number or content). In some embodiments, the biological signature is exosomes profile (either number or content). In some embodiments, the biological signature is circulating cells profile. In some embodiments, the biological signature is circulating tumor cells profile. In some embodiments, the biological signature is circulating immune cells profile. As used herein, the term “profile” is intended to encompass any variation of the determined entity including the presence or absence, as well as the type (e.g., genotype), amount, percentage or difference in expression, as long as it is suitable for prediction of the response to therapy.

Methods of performing expression profiling are well known in the art. RNA expression can be assayed by any known method including, polymerase chain reaction (PCR), real-time PCR, quantitative PCR, digital PCR, microarray, northern blotting, and sequencing. In some embodiments, the expression profiling comprises PCR. In some embodiments, the expression profiling comprises hybridization to a microarray. In some embodiments, the expression profiling comprises sequencing. In some embodiments, the sequencing is next-generation sequencing. In some embodiments, the sequencing is deep sequencing. In some embodiments, the sequencing is massively parallel sequencing. Methods of sequencing are well known in the art, and apparatuses for sequencing are commercially available. Any known method of sequencing may be used in accordance with the method of the invention.

Protein expression can be assayed by any known method including, an immunoassay, immunoblotting, immunohistochemistry, FACS, ELISA, Western blotting, proteomics arrays, proteome sequencing, proximity-extension assay (PEA)-based assay, aptamer-based assays, multiplex assay and mass spectrometry. In some embodiments, the expression profiling comprises hybridizing to a proteomics array. In some embodiments, the proteomics array is an antibody array. In some embodiments, the expression profiling comprises whole proteome sequencing. In some embodiments, the expression profiling comprises targeted mass spectrometry. In some embodiments, the expression profiling comprises untargeted mass spectrometry. In some embodiments, the expression profiling comprises shotgun proteomics using mass spectrometry. In some embodiments, the expression profiling comprises top-down mass spectrometry. In some embodiments, the expression profiling comprises bottom-up mass spectrometry. In some embodiments, the expression profiling comprises data-independent acquisition (DIA) mass spectrometry. In some embodiments, the expression profiling comprises data-dependent acquisition (DDA) mass spectrometry. Proteome/proteomics arrays are well known in the art and are commercially available. Examples of proteomics arrays include, but are not limited to, the Proteome Profiler Array of R&D Systems, the CP Human Proteome array of Creative Proteomics, RPPA (reverse phase protein array), the human Kiloplex Quantitative Proteomics array of RayBiotech, Olink Target 96, Olink Explore 96 and the Membrane Proteome Array of Integral Molecular.

In some embodiments, at step 104, a preprocessing stage may take place, comprising at least one of data cleaning and normalizing, data quality control, and/or any other suitable preprocessing method or technique.

Biological data derived from clinical samples may suffer from variations that can arise due to different sample collection or sample preparation procedures, due to quantification inaccuracies, due to batch effects, and/or due to any other technical bias that may lead to mistakes in the analysis. Therefore, in some embodiments, preprocessing may comprise a quality control step wherein at least some biological signatures may be removed based, at least in part, on a measurability-parameters of proteins expressed in the biological signature.

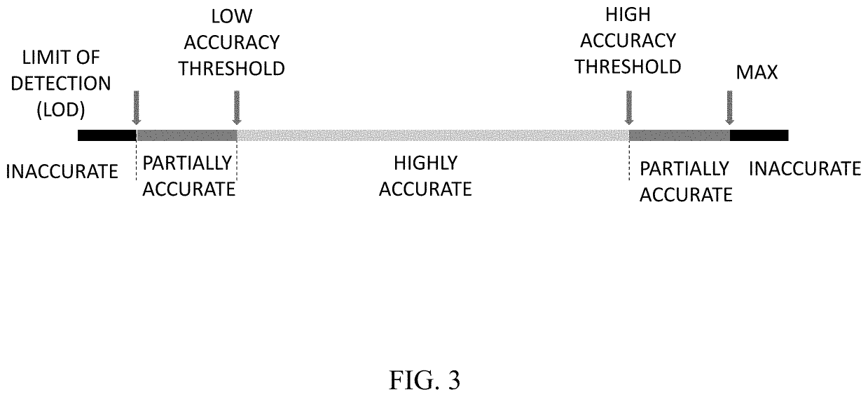

In some embodiments, quality control and/or data cleaning and/or data normalization may comprise any one or more of the following:
Data transformations: For example, a log 2 transformation, Z-score transformation, median subtraction.
Statistical tests: key statistical measures, such as median, average, the first quartile of the dataset (Q1), the third quartile of the dataset (Q3), variance, standard deviation or coefficient of variation (cv), are calculated in order to assess the data quality.
Data visualization: Enables a better understanding of the data, whether the data are normally distributed, or whether there are any technical biases, batch effects or any outliers that behave substantially different from the rest of the samples.
Evaluation of data quality: Includes a step of defining which data should be included/removed/normalized in the analysis, thereby generating a new output containing only the desired and normalized results.
Handling quality control data issues: In specific cases, mostly due to technical biases, extremely different samples are considered for exclusion. In case of batch effects due to technical reasons, batch effect removal algorithms and/or data normalization can be applied.
Batch effect removal: Can be done in different ways. Non-limiting examples are: using batch effect removal algorithms (e.g., limma); subtracting component/s in principal component analysis (PCA); median subtraction; Z-scoring; running the same reference samples in different batches (“bridging samples”) and correcting based on their values.
Handling data below limit of detection (LOD): The approach for dealing with values below the LOD level can be done by data imputation: As a non-limiting example, T₀ or T₁ values that are below LOD can be assigned the LOD level of the examined protein. In case both time points are imputed, the T₁/T₀ ratio equals to 1, and after log 2 transformation it equals to 0; in some data analyses, it can be assigned as ‘not a number’ (NaN) value instead. Other approaches for data imputation can be also used. FIG. 3 is a schematic illustration of a quality control process which may be used to assess measurements below a limit of detection (LOD) threshold and/or above a maximum threshold.
Missing values or 0 values handling: Proteins which have missing (NaN), below LOD value or 0 values in less than 0-100% of the samples are filtered. Alternatively, missing (NaN), below LOD value or 0 values can be imputed by any other imputation method. Following any data imputation, some QC steps may be repeated.

Data normalization: If needed, the data are normalized prior to the bioinformatic analysis. Data normalization can be performed at any level, e.g., protein-level, batch level, etc.

In some embodiments, at step 106, differential expression values may be calculated with respect to each pair of biological signatures. In some embodiments, with respect to the case of biological signatures that are protein expression profiles, the present disclosure provides for calculating the relations (e.g., a level of difference in expression values between each biological signature in a pair of signatures associated with a subject, e.g., a difference in, and/or a ratio of, expression values between biological signatures at at-least two time points relative to the therapy, e.g., a T₁/T₀ ratio. In some embodiments, this analysis does not take into account any biological function of the proteins and/or any known interactions between the proteins. In some embodiments, T₁/T₀ ratio is a numerical value determined by calculating the ratio of on-treatment and baseline values (pre-treatment). The T₁/T₀ ratio may be used to predict responsiveness or non-responsiveness of the patient to the cancer treatment.

In some embodiments, additional, other, and/or alternative sets of values may be calculated, associated with, e.g., biological processes, clinical data, and/or protein-interaction driven analysis between T₁/T₀ signatures.

In some embodiments, at step 108, one or more feature selection, feature extraction, an ensemble process, and/or dimensionality reduction steps may be performed with respect to the value sets.

In some embodiments, feature selection and/or dimensionality reduction steps may be performed, to reduce the number of variables in each sample pair and/or to obtain a set of principal variables, e.g., those variables that may have significant predictive power such as protein expression levels. Accordingly, in some embodiments, a feature selection and/or dimensionality reduction step may result in a reduction of the number of proteins in each biological signature and/or set of values. In some embodiments, dimensionality reduction selects principal variables, e.g., proteins, based on the level of response predictive power a protein generates with respect to the desired prediction. In some embodiments, the dimensionality reduction generates a new feature or features that can be predictive for response. In specific embodiments, the dimensionality reduction involves regarding all or some feature values as vector components and calculating its norm.

In some embodiments, any suitable feature selection and/or dimensionality reduction method or technique may be employed, such as, but not limited to:
ANOVA with S₀ parameter: Analysis of variance with an additional parameter (S₀) that controls for the relative importance of features based on resulted test p-values and difference between the group means (see, e.g., Tusher, Tibshirani and Chu, PNAS 98, pp 5116-21, 2001).
Scalable EMpirical Bayes Model Selection (SEMMS): An empirical Bayes feature selection method which applies a parsimonious mixture model to identify significant predictors (see, e.g., Bar, Booth, and Wells. A scalable empirical Bayes approach to variable selection in generalized linear models, 2019).
L2N: A method for differential expression analysis that uses a three-component mixture model. The model consists of two log-normal components (L2) for differentially expressed features, one component for under-expressed features and the other for overexpressed features, and a single normal component (N) for non-differentially expressed features (see, e.g., Bar and Schifano. Differential variation and expression analysis. Stat 8, e237, doi:10.1002/sta4.237, 2019).
Genetic algorithms: A family of heuristic optimization algorithms that employ organic evolutionary techniques such as random mutations, recombination, and natural selection as methods for achieving optimal configurations (see, e.g., Popovic, Sifrim, Pavlopoulos, Moreau, and Bart De Moor. A Simple Genetic Algorithm for Biomarker Mining. 2012).
Naïve classifier: The naïve classifier evaluates a response score by reducing the dimension to a single score. This is performed by regarding all features (e.g., specific profiles such as protein expression levels) as component of a vector and calculating its norm. The dimension reduction reduces the possible risk of an over-fitting. In some embodiments, the vector components are normalized according to the typical component value among patients that belong to the same response group (e.g., responders), such that the normalized norm quantifies the amount of deviation from the typical respective class value. In additional embodiments, the naïve classifier enables training using data of subjects that belong only to part of the response groups.

In some embodiments, at step 110, a training dataset for training a machine learning model of the present disclosure may be constructed, comprising sets of values representing relations (e.g., ratios or difference in expression values) of the biological signatures at multiple time points relative to the therapy, with respect to at least some of the subjects in the cohort.

In some embodiments, a training dataset of the present disclosure may comprise additional information for training of the machine learning model, such as clinical, demographic, and/or physical information with respect to at least some of the subject in the cohort. For example, in some embodiments, such data may include characteristics obtained from the diseased tissue itself (e.g., from a tumor of a cancer patient). In some embodiments, such data may include, but is not limited to: demographic information (ex, age, ethnicity); performance status; hematological and chemistry measurements; cancer disease history, e.g., date of cancer diagnosis, primary cancer type and stage, disease biomarkers (e.g. PD-L1), disease treatment history, histology, TNM stage, assessment of measurable lesions, time of tumor progression, site of recurrence, proposed treatment; general medical history, including smoking history and drinking habits, background diseases including hypertension, diabetes, ischemic heart disease, renal insufficiency, chronic obstructive pulmonary disease, asthma, liver insufficiency, Inflammatory Bowel Disease, autoimmune diseases, endocrine diseases, and others; family medical history; genetic information, e.g. mutations, gene amplifications, and others (e.g. EGFR, BRAF, HER2, KRAS, MAP2K1, MET, NRAS, NTRK1, PIK3CA, RET, ROS1, TP53, ALK, MYC, NOTCH, PTEN, RB1, CDKN2A, KIT, NF1); physical parameters, e.g., temperature, pulse, height, weight, BMI, blood pressure, complete blood count including all examined parameters, liver function, renal function, electrolytes; medication (prescribed and non-prescribed); relative lymphocyte count; neutrophil to lymphocyte ratio; baseline protein levels in the plasma (e.g. LDH); and/or marker staining (e.g. PD-L1 in the tumor or in circulating tumor cells). In some embodiments, a change in response to the specified therapy in one or more of the above information may be analyzed and provided for training of the machine learning model.

In some embodiments, one or more annotation schemes may be employed with respect to the training dataset. Accordingly, in some embodiments, a training dataset for a machine learning model of the present disclosure may comprise a plurality of sets of T₁/T₀ ratios or difference in expression values with respect to at least some of the subjects in the cohort, wherein at least some of these sets of values may be annotated with category labels denoting a response and/or outcome of the treatment in the respective subject. In some embodiments, such annotation may be binary, e.g., positive/negative, and/or expressed in discrete categories, e.g., on a scale of 1-5. In some embodiments, a binary value category label may be expressed, e.g., as ‘yes/no,’ responsive/non-responsive,′ or favorable/non-favorable response.′ In some embodiments, discrete category labels and/or annotations may be expressed on a scale, e.g., ‘complete response,’ partial response,′ ‘stable disease,’ progressive disease,′ pseudo-progression,′ and ‘hyper-progression disease.’ In some embodiments, additional and/or other scales and/or thresholds and/or response criteria may be used, e.g., a gradual scale of 1 (non-responsive) to 5 (responsive). In some embodiments, category labels may be associated with adverse or any other secondary effects or response by a patient, e.g., therapy side-effects.

In some embodiments, additional and/or other annotation schemes may be employed. In some embodiments, the training dataset may be annotated with, e.g., patient demographic and/or clinical data as detailed above. In some embodiments, the training dataset may be annotated with overall response rate. In some embodiments, the training dataset may be annotated with progression-free survival rate. In some embodiments, the training dataset may be annotated with duration of response rate.

In some embodiments, at step 112, a machine learning model may be trained on the training dataset constructed in step 110. In some embodiments, any suitable machine learning algorithm or combination of methods may be employed, including, but not limited to:
Support Vector Machine (SVM): A nonparametric model which finds the optimal separating hyperplane that discriminate between different classes. It can perform linear or non-linear classification.
Penalized Logistic Regression (PLR)—a logistic model for regression that imposes a penalty to reduce the impact of certain features.
Generalized linear model (GLM): a generalization of linear regression that unifies statistical models such as linear regression, logistic regression and Poisson regression. GLM extends linear regression by (1) supporting response variables with error distributions other than the normal distribution (2) a non-linear relationship between the predictors and the response variable.
Random forest (RF): involves in the generation of multiple decision trees that consist sequences of decision rules for protein expression values. To avoid over-fitting, these trees may be pruned. Each tree is constructed by randomly selecting different samples.
eXtreme Gradient Boosting (XGB): a gradient boosted decision trees-based classification and regression algorithm. The decision trees are built one at a time, and each new tree corrects the error of the previously trained decision tree.

In other embodiments, machine learning model may be trained based on statistical measures, i.e., variance, median, mean, average and the same.

In some embodiments, at step 114, the machine learning model results with a classifier a target set of T₁/T₀ relations (e.g., ratios or difference in expression values) suitable for predicting a response in a target patient and receiving a similar treatment as the patient cohort.

In some embodiments, at an inference step 114, a trained machine learning model of the present disclosure may be applied to target data, e.g., a target set of T₁/T₀ relations (e.g., ratios or difference in expression values) with respect to a target patient with similar phenotype and receiving a similar treatment as the patient cohort. In some embodiments, the inference of the trained machine learning model on the target data produces a therapy response prediction or response probability.

In some embodiments, the prediction is for side-effect or adverse event. In some embodiments, the prediction is for overall survival rate. In some embodiments, the prediction is for progression-free survival rate. In some embodiments, the prediction is duration of response rate. In some embodiments, the prediction is for pseudo-progression. In some embodiments, the prediction is for hyper-progression. In some embodiments, the prediction is for progression of the disease. In some embodiments, a prediction according to the present disclosure may be further supported and/or supplemented with a Differentially Expressed Protein Identification and/or Differentiating Biological Processes analyses, as further detailed herein below.

In some embodiments, at step 116, a therapy course with respect to the target patient may be administered, adjusted, and/or modified based, at least in part, on the inference step 114. In some embodiments, such therapy adjustment may include prescribing a subsequent and/or supplementary therapy for the target patient.

### Differentially Expressed Protein Identification

In enrichment analyses, including enrichment analyses, some network-based analyses when focusing on a subset of the features, or for providing personalized potential targets for therapeutic intervention, as defined below, one needs to first identify the DEPs between the examined groups (e.g., responders vs. non-responders).

The term “Differentially Expressed Proteins” (DEPs) refers to proteins whose distribution (of expression level or change between two timepoints, e.g., T₀ and T₁) differs between responders and non-responders (and possibly other groups, e.g., stable disease patients), including any difference in distribution detectable by numerical measures (e.g., t-test, ANOVA, Kolmogorov-Smirnov test). In some cases, DEPs are defined as proteins who's median, mean, variance or other statistical measure differ between responders and non-responders. In some cases, DEPs are defined as proteins whose distribution differs between responders and non-responders without alteration of the mean or the median, however, the difference can be evaluated by statistical tests. In some cases, DEPs are defined as proteins that in at least one patient, do not follow the respective protein distribution among a specific subgroup (i.e., responders or non-responders).

Identifying the DEPs is an optional step, as some tools do not require a list of DEPs, but rather rely on a selected measure that is calculated for all the proteins in the proteomic profile, such as fold change between the two examined groups (i.e. responders and non-responders), or the p-value of the t-test.

The terms “protein profile”, “protein expression profile” and “proteomic profile”, used herein interchangeably, refer to the expression level of a protein or a list of proteins, such as cytokines, growth factors and other proteins expressed in plasma, CSF or other body fluids, or tissues, at a certain time point. The number of proteins measured may vary between 1 and 20,000. A protein profile may be used to diagnose a disease, condition, or syndrome and to determine the odds of treatment response. In some embodiments, the profile comprises at least 1, 2, 3, 4, 5, 6, 7, 8, 9, 10, 15, 20, 25, 30, 35, 40, 45, 50, 60, 70, 80, 90, 100, 500, 1000, 2000, 5000, 7000 or at least 20000 proteins. Each possibility represents a separate embodiment of the invention. In some embodiments, the protein profile is absolute protein expression. In other embodiments, the protein expression profile is normalized or relative expression profile.

Differentiating Biological Processes

Differentiating Biological Processes can be deciphered by using DEPs as an input. Another method is by running per sample pathway enrichment and then aggregating the enrichment results.

The term “Differentiating Biological Processes” (DBP) refers to biological processes that occur in either the responding patients or the non-responding patients.

Differentiating biological processes may be provided based on different databases, such as the KEGG pathways analysis (https://www.genome.jp/kegg/) and gene ontology (GO) analysis (geneontology.org).

Pathway Enrichment Analysis

Proteomic analysis at the pathway level may imply biological processes that are affected by the treatment. The aim of this step is to translate host-response related changes at the protein expression level to changes at the biological process level. Thus, the DEPs identified can be used as an input for the pathway enrichment analysis.

Pathway enrichment analysis translates the proteomic changes into a list of estimated biological processes which were either down- or up-regulated in the body in response to the cancer therapy treatment. The analysis can identify DBPs, which are biological processes enriched in each group. The output of the enrichment analysis is a list of DBPs and the proteins that are involved in each one of them.

Network Analysis

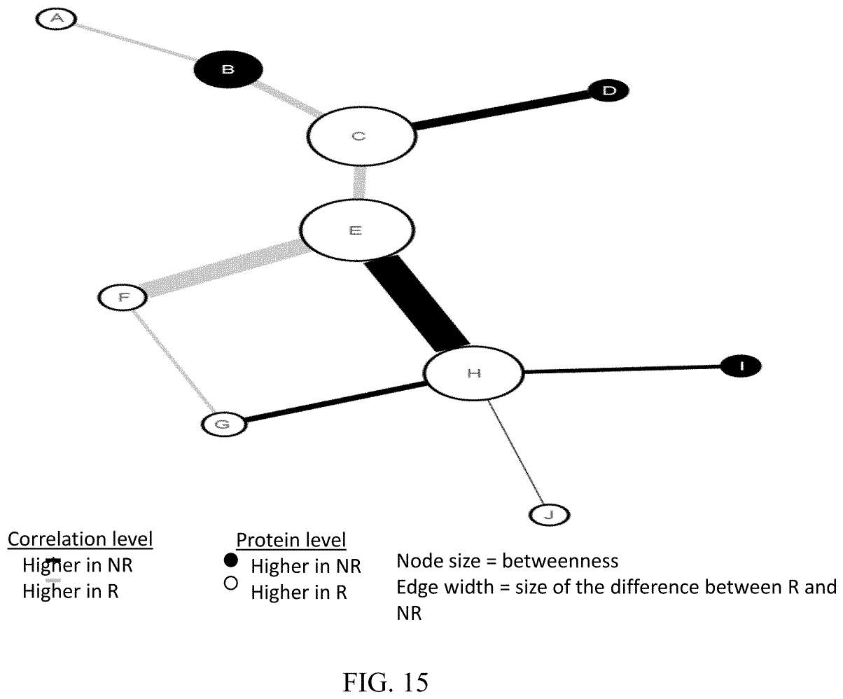

An additional level of analysis that is based on the proteomics data involves studying the co-changes in the expression levels of several proteins together; proteins that display a correlation (either a negative or a positive correlation) between their proteomics profiles may indicate a potentially interesting biological relation. Correlations between proteins may occur due to various reasons, such as a common regulator that affects both proteins (e.g., a transcription factor, a phosphatase, etc.) or that one of the proteins is a regulator of the other. Based on the correlations between the expression levels of different proteins one can construct protein networks. Protein networks may differ between two different conditions (e.g., responders and non-responders) and therefore studying these differential networks (FIG. 14) may decipher biological mechanisms underlying the different phenotypes (e.g., resistance to therapy). An example for such differential network is displayed in FIG. 15.

Examination of the differential network between responders and non-responders may reveal novel mechanisms that are associated with resistance to therapy and may be used for classifier training. Network analysis can be helpful for feature engineering. For instance, such analysis can aid in pinpointing features that are changing together; an engineered feature that captures this relation may be predictive by using any mathematical relation between two or more proteins. FIG. 16 demonstrates a non-limiting example of a predictor based on correlations between protein pairs. Two proteins showing differentially correlated fold-change values between responders and non-responders were inspected. FIG. 16A—Correlation between responders (triangle) and non-responders (circles) was positive (R=0.37). The dashed line shows a linear fit to the responder values. FIG. 16B—The residuals (i.e., distances of each point from the linear fit in FIG. 16A) of two protein pairs was calculated and used as an input for SVM-based classifier. The resulting predictor achieved a ROCAUC of 0.765. In addition, network analysis can potentially identify novel approaches for intervention, which could be highly valuable for pharmaceutical companies.

### Personalized Potential Targets for Intervention

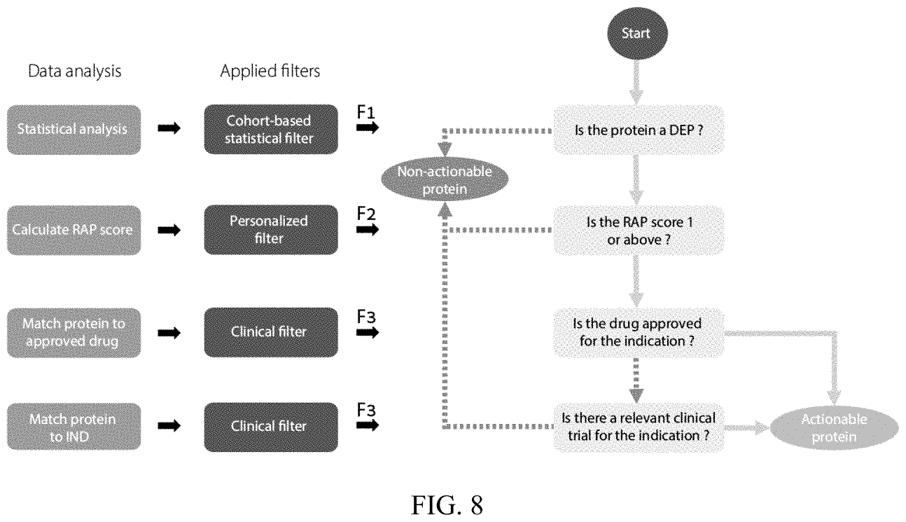

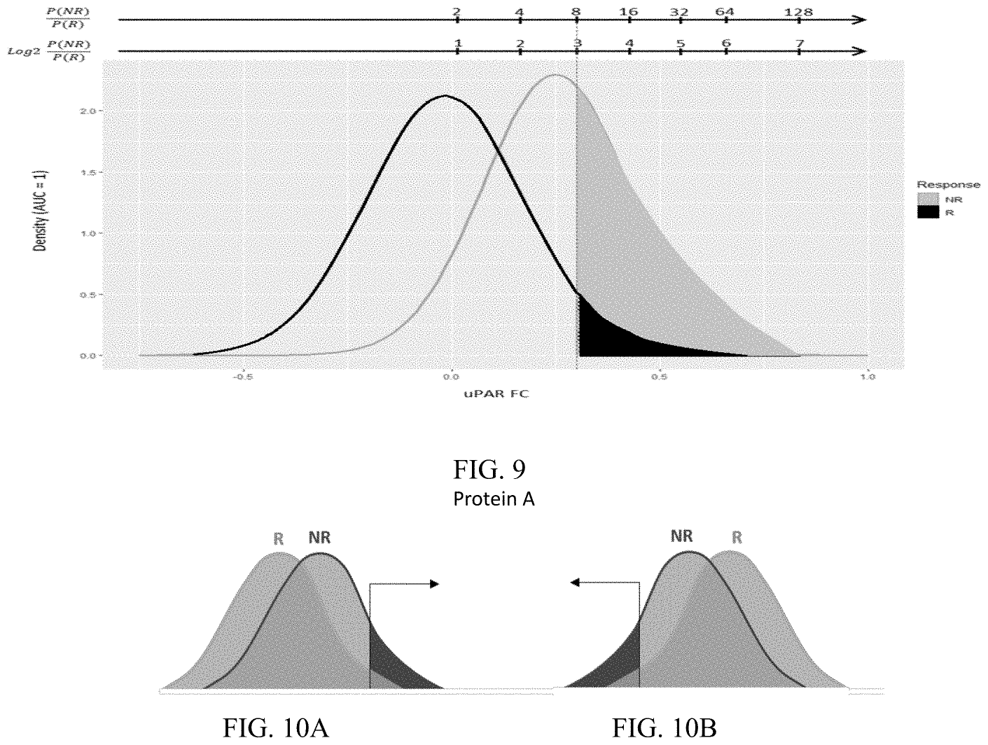

In another aspect, the invention provides an analysis of personalized potential targets for intervention, using a 3-filter approach. The three-filter approach can be visualized in a decision-tree like flowchart (FIG. 8) that starts with the first filter, which makes use of the cohort strength, keeping only the DEPs. A protein that is not a DEP does not continue to the next filter and is considered a non-actionable protein. The DEPs used for this analysis may be similar or may differ from the DEPs provided for training the machine learning model. The second filter focuses on the patient's specific proteomic profile of the DEPs, keeping only the patient's resistance associated proteins (RAPs) based on a RAP score (described in detail below). A DEP that had a RAP score below the threshold (e.g., RAP score of 1) is considered not relevant to the specific patient and is filtered out. Next, the third filter for associating a RAP to a drug or investigational new drug, is applied. The third filter may include two options; if the RAP has an approved drug (e.g., specific for the indication of interest, or a different indication), then the RAP is considered an actionable protein. Alternatively, if there is a clinical trial associating the RAP and a candidate drug (e.g., for the indication of interest), the RAP may also be considered as an actionable protein.

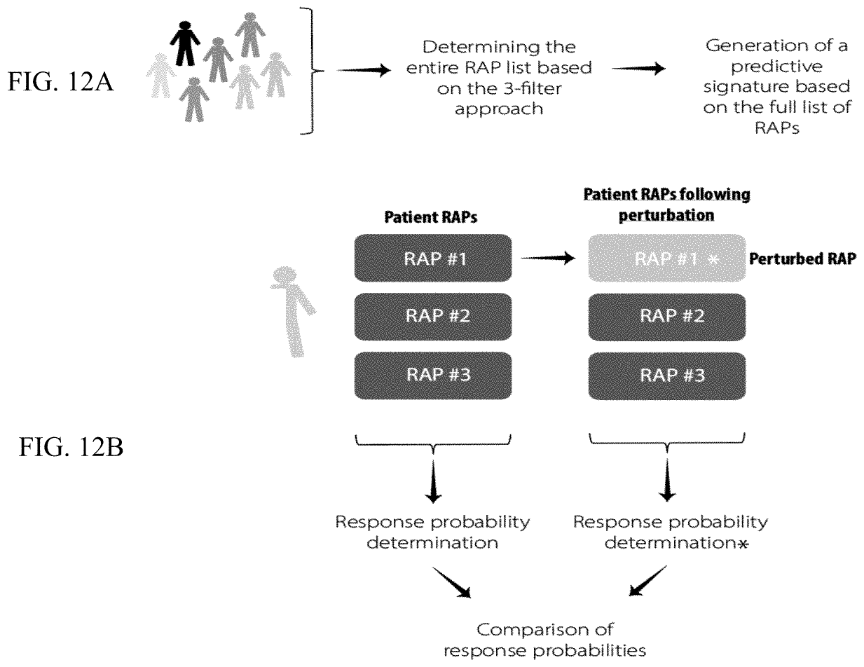

The analysis provided herein may further include a simulation step wherein the RAP (or multiple RAPs) expression value of the patient is modified towards a balanced value that may follow the therapeutic intervention. Next, another prediction analysis is performed, aiming to assess the effect of the change in the protein expression value. This may help the physician in deciding which RAP to select for the patient (multiple RAPs that are potential targets for intervention may be received for a selected patient). FIG. 12 demonstrates a non-limiting simulation of a single RAP perturbation (although simulation of several RAPs perturbation may be also performed). A predictive signature that is based on the list of RAPs of the entire cohort is first generated (FIG. 12A). For a given patient, a specific RAP, or combination of RAPs, is balanced and the baseline response probability is then compared to the perturbed response probability (FIG. 12B).

Cohort-Based Statistical Filter

It is first essential to identify DEPs. As described herein, DEPs are proteins whose levels change between responders and non-responders and may also change between T₀ and T₁ in responders and non-responders. In some cases, DEPs are proteins whose median differ between responders and non-responders. In some cases, DEPs are proteins whose variance differ between responders and non-responders. In other cases, DEPs are proteins whose average differ between responders and non-responders. This analysis narrows down the number of proteins, yielding a list of proteins that cohort-wise display differences between the two or more classes, and thus potentially play a role in the resistance or the response to treatment. The DEPs may be identical or may be independent of the DEPs identified for the machine learning aspects of the invention.

Personalized Filter

The list of proteins from the first filter (the cohort-based statistical filter) is examined in a patient-specific manner using the personalized filter and the resistance-associated protein (RAP) score.

RAPs are defined per patient. In some embodiments, a patient's RAP is a protein whose levels or fold-change is deviant from the respective protein distribution in one of the response group. The deviation may be quantified by numerical means, either by using the levels in multiple response group (e.g., both responders and non-responders), or the distribution among a specific response group (e.g., responders or non-responders or stable disease patients). For a non-limiting example, the expression distribution of each DEP in the entire cohort can be examined per response class (meaning—a distribution for responders and a distribution for non-responders are generated separately). In this case, a probability density distribution can be extracted for each group distribution, with a total area under the curve of 1. For each patient, the area (or other measures, such as the height in each of the response group distributions) of the tail above or below the DEP expression level (e.g., selecting above or below may depend on the order of the non-responder and responder medians, as described in detail below) is calculated for each distribution (Figure). Next, the RAP score may be calculated per DEP for each patient, such as based on the non-limiting Equation 1:

$$R A P score = {log}_{2} \frac{P ( NR )}{P ( R )} \qquad ( Equation 1 )$$

where P(NR) is the probability that the protein can be attributed to the non-responder distribution and P(R) is the probability that the protein can be attributed to the responder distribution. The RAP in this example is a probabilistic measure for the distance of the DEP expression level from responder group distribution. A high RAP score designates a protein with a high probability of being associated with the non-responder distribution rather than to the responder distribution.

The RAP score may be expressed as a simplification of the

$${log}_{2} \frac{P ( NR )}{P ( R )}$$

measure, whereby the RAP score may be presented in a five-bin format using 5 degrees that represent the fold increase in the odds. A score of 5 or higher may be grouped to a score designated as 5+.

For each patient, the DEPs following the first filter are examined, and the RAP score is calculated per each DEP. Optionally, the threshold for passing the personalized filter can be defined (e.g., a RAP score above 1.0). This list of the patient's RAPs continues to the next filter.

Directionality Calculation

A RAP score above the defined threshold may indicate that the protein can be attributed to the non-responder distribution (note that respectively, a reverse high RAP score can attribute the RAP to the responder distribution). In a case where there is a difference in medians between responders and non-responders, the selection of the direction of the tail of the area under the curve can be based on the order of the distribution medians. If the median of the non-responder group is higher than the median of the responder group, the area selected for the calculation is the tail to the right of the DEP expression level of the patient (FIG. 10A). If the median of the non-responder group is lower than the median of the responder group, the areas under the left tail of the distributions are used for the DEP expression level of the patient (FIG. 10B). This way, proteins that are more likely to be attributed to the non-responder distribution have values above 1.0 (log 2 scale), regardless of the DEP direction in the cohort (higher in responder or higher in non-responder). DEPs that are more likely to be attributed to the responder group have RAP scores between 0 and 1 (log 2 scale). In cases where medians are not differential but other aspects of the distribution change, i.e., variance or average, a relative probability at a given range (not necessarily the tail) may also be used.

The Effect of the DEP Statistics on the Distribution of the RAP Scores

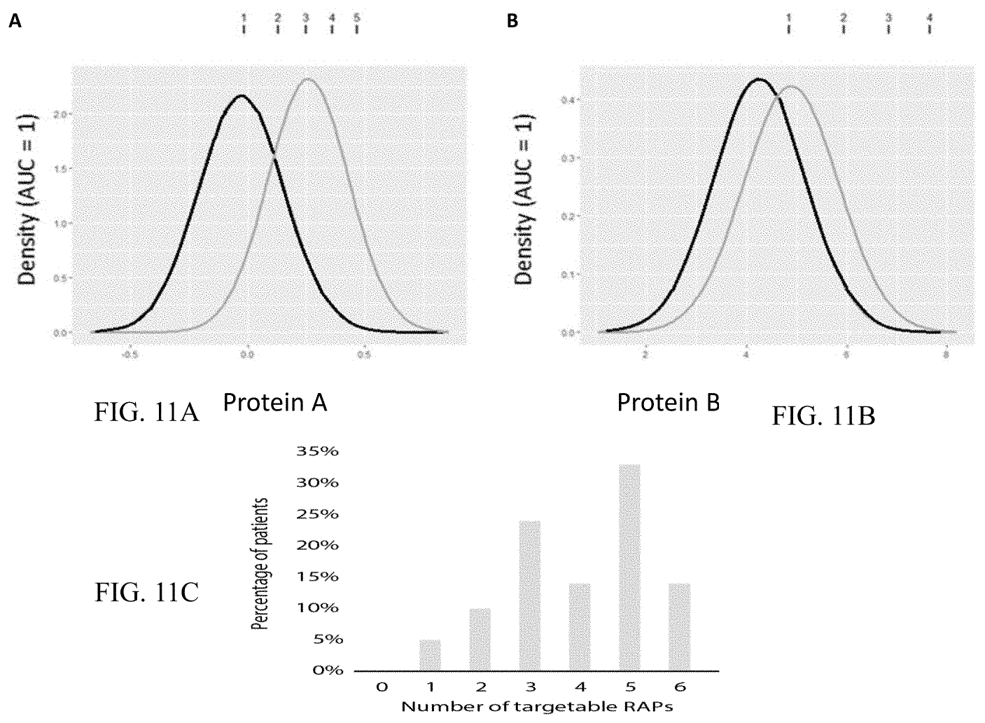

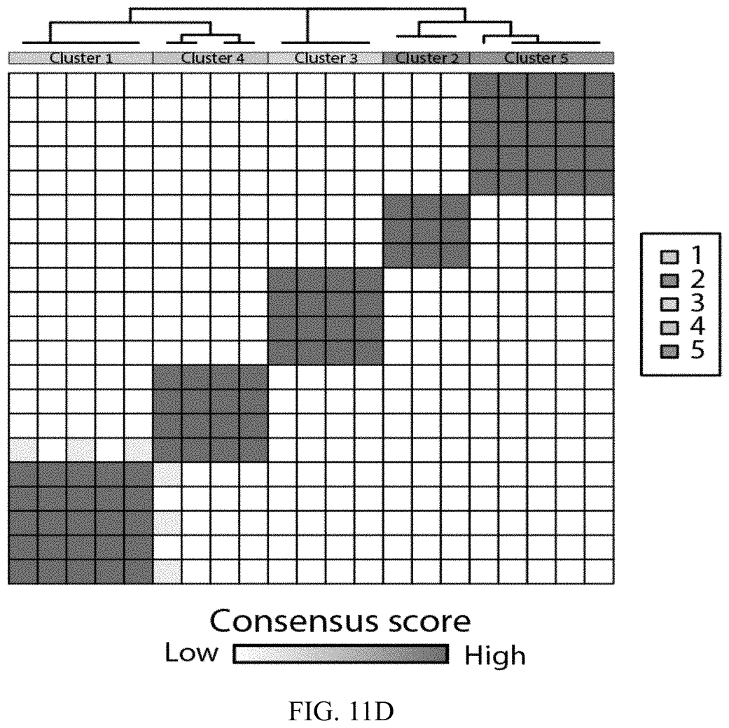

Since the RAP score is based on the responder and non-responder distributions of the entire cohort, the difference between the responder and non-responder distributions affects the RAP score distribution (FIGS. 11A-11D). A DEP with a large difference between responders and non-responders displays smaller gaps between RAP scores, indicating that patients are more likely to have high RAP scores (FIG. 11A and FIG. 11B) compared to a DEP with a smaller difference between responders and non-responders, which displays larger gaps between RAP scores, indicating that patients are less likely to have high RAP scores (FIG. 11C).

RAP-Based Clusters

The RAP score can be further used to identify groups of non-responders that share similar RAP profiles. For this analysis different clustering algorithms can be used. A non-limiting example is the use of consensus clustering algorithm, which finds the most robust clusters of samples following multiple iterations of clustering, can be used. An example for that is displayed in FIG. 11D. The different clusters can then be further characterized to examine enrichment of various clinical parameters. For example, cluster #5 was enriched with patients who recently quit smoking (Fisher exact test p-value=0.027).

Clinical Filter

Following the first two filters described above, a list of personal RAPs is generated for the patient. In the current filter (FIGS. 12A-B), potential drugs or investigational new drugs (INDs) that target the patient's RAPs are identified. The clinical filter may include the following steps. First, drugs/INDs that target each RAP are searched in a suitable database and are associated with the RAP. Next, various clinical filters may be applied following data collection and analysis; this may include biological reasoning-based examination layer, mode of action related layers (such as direct/indirect; specific/non-specific; directionality match) and drug clinical relevance related layers (such as drug development status/clinical relevance). RAPs associated with drugs/INDs based on the applied filters can be considered as potential intervention targets for the patient. Alternatively, they may be considered as a basis for potential collaboration with pharmaceutical companies.

### EXPERIMENTAL RESULTS

### Example 1- Melanoma Cohort

The present inventors conducted an experiment to test the prediction power of a machine learning model of the present disclosure.

The training dataset of the experiment comprised biological samples from melanoma patients. Response to treatment for each patient was determined based on either response evaluation criteria in solid tumors (RECIST) estimation or clinical benefit evaluation. Patients with progressive disease (PD) were categorized as non-responders (NR). Patients who displayed partial response (PR) or complete response (CR) were classified as responders (R). Patients with stable disease (SD) status were categorized as SD patients.

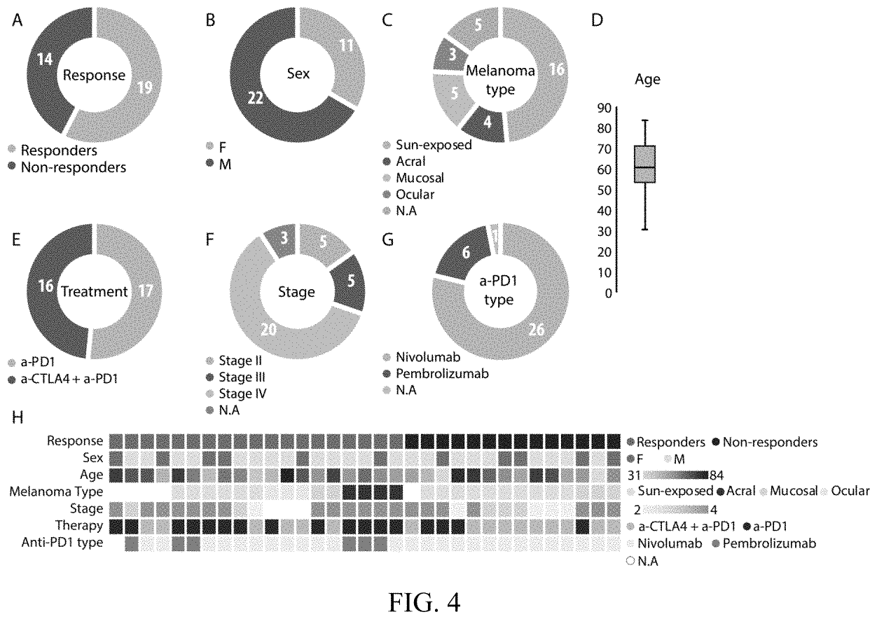

In the bioinformatic analysis, SD samples were excluded in order to focus on the two more extreme and distinct groups of responders and non-responders. Excluding the SD group is sometimes performed by other research groups, since it acts differently from the R and NR groups. Ultimately, the dataset included 33 samples for the analysis. Clinical parameters of the training dataset are displayed in FIG. 4.

For each patient, plasma samples pre- (T₀) and early on- (T₁) treatment were collected. Using antibody array technology by RayBiotech (see, Wilson, J. J. et al. Antibody arrays in biomarker discovery. Adv Clin Chem 69, 255-324, doi:10.1016/bs.acc.2015.01.002 (2015)), the proteomic changes during anti-PD1 or anti-PD1 combined with anti-CTLA4 treatment were profiled. A total of 400 proteins were profiled per sample. A predictive biological signature for response to treatment was extracted based on a log 2 of T₁/T₀ ratios (log fold change) for each protein.

The data were processed with the following steps. First, T₀ or T₁ values below limit of detection (LOD) were assigned the LOD value. Following log 2 fold change transformation, proteins with both T₀ and T₁ LOD values had a value of log 2 fold change of 0. The data was filtered to keep proteins with 0 values (proteins which both T₀ and T₁ values were below limit of detection) in less than 50% of the samples. Altogether, this filtration step resulted in 330 valid proteins for the downstream analysis. Additionally, in the QC analysis, a large variation between the samples was observed, while not all were centered at the 0 value. Therefore, the data were normalized by subtracting the overall median from each sample.

In order to identify a proteomic signature that would enable the prediction of response, patients with relatively large or small overall variability were excluded from the training set. Thus, the first step consisted of differential expression (based on log 2 fold change values) analysis between the group of responders (n=5) and non-responders (n=8).

To identify response predictive proteins, the L2N method was applied to identify differentially expressed proteins (DEPs) between responders and non-responders. One advantage of using this differential expression approach to identify predictive proteins is that it relies on normal models for continuous data, which are more powerful than binary classification method, and thus require a smaller sample size to obtain the set of predictors. The second advantage of using this approach is that it reduces the chance of overfitting because it provides a separation between the two steps, the first being the process of fitting a model to find differentially expressed proteins, and the second step involves fitting a logistic model of the response status of patients using only the differentially expressed proteins as predictors. Using this approach, 10 differentially expressed proteins were identified. Response to treatment was determined based on clinical benefit (i.e., responders or non-responders).

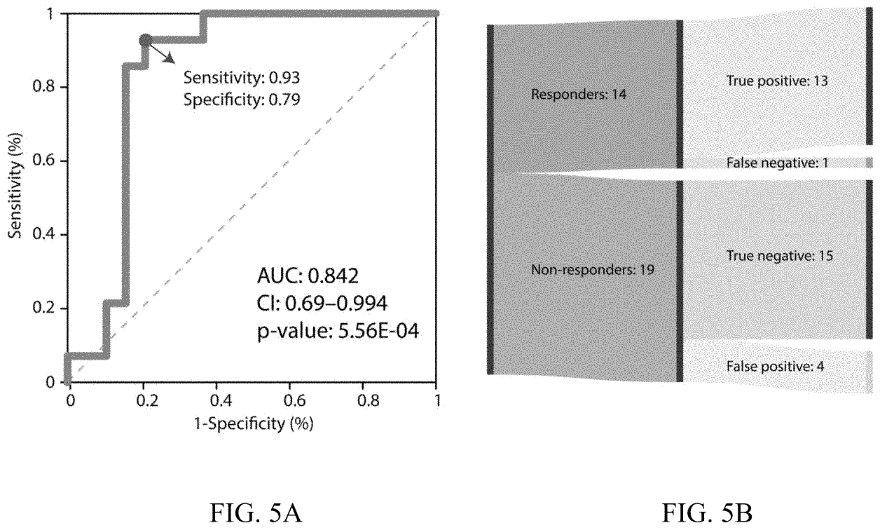

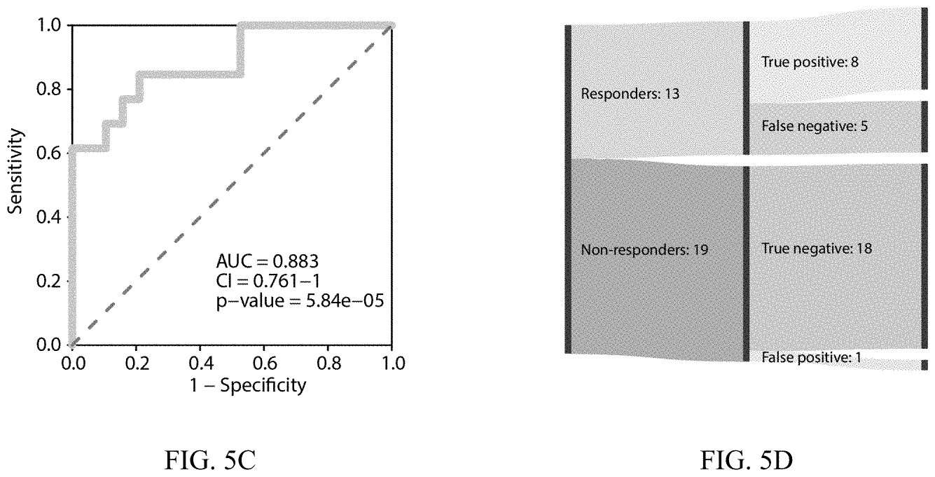

A logistic regression model (GLM) was generated using the 10 selected proteins as predictors and the true response status as the dependent variable. A good prediction was obtained with an area under the curve (AUC) of 0.84 in the receiver operating characteristics (ROC) plot on the entire dataset of 33 patients (FIG. 5A), and a total of 6 misclassifications. Note that three of the misclassified patients were among the 13 samples used in the first step. This suggests that there is a low probability for overfitting. Among the 20 left-out patients, the predicted probability of responding to the treatment of 17 patients (85%) was in agreement with their actual status. A point in the ROC plot was selected with at least 90% sensitivity and the maximal specificity at this level, which resulted in sensitivity and specificity of 0.93 and 0.79, respectively (FIG. 5B).

Another approach for discovering a predictive signature for response to treatment based on host-response using the log fold change values was done using linear Support Vector Machine (SVM) algorithm. In this approach, response to treatment was determined based on response evaluation criteria in solid tumors (RECIST) estimation. Using this approach, several single protein predictors were identified, and the top 25 protein predictors showed a varied AUC between 0.7 to 0.822. To maximize prediction ROC AUC with a minimal number of proteins, models of multiple proteins were also generated. The best prediction was obtained in a model comprised of 3 proteins that yielded a ROC AUC of 0.88. (FIG. 5C-5D).

To validate the results of the single protein and the multi-protein classifiers, a different cohort of patients was used. This validation cohort dataset comprised of biological samples from 14 patients, from which plasma samples pre- (T0) and early on- (T1) treatment were collected, and the proteomic changes during ICI treatment were profiled, as previously described. Validation of the single protein classifier obtained previously on the validation cohort have demonstrated that not all the single protein-based models generalized well with the validation cohort dataset. Further examination of the validation of the multi-protein classifiers have demonstrated that the 3-protein model showed good predictive results with an AUC of 0.85.

### Enrichment Analysis

Previous studies have shown that a combination therapy has improved response rates of immunotherapy. In this cohort of melanoma patients, the differences between the anti-PD1 monotherapy and combination therapy of anti-PD1 and anti-CTLA4 were tested by analyzing the main biological pathways and proteins that underlie these treatment modalities, and identifying differentiating biological processes. In contrast to the classifier analysis, which does not consider the biological function of the proteins that are part of the classifier, this analysis aims to characterize the host response in the context of differentiating biological processes (DBPs) that change upon treatment and are different between responders and non-responders. This exploration can then be used as a basis for the identification of driver proteins that can be potential targets for intervention as part of a combination therapy with immunotherapy. To this end, the proteomics data was analyzed using the MetaCore tool from Clarivate Analytics. The great advantage of using this tool is the highly curated database on which the protein maps are based on.

Before performing the enrichment analysis that can identify the DBPs, a statistical test was applied to select the differentially expressed proteins (DEPs; proteins whose levels change between responders and non-responders, and/or between T₀ and T₁ in responders and non-responders), that may serve as an input for the enrichment analysis.

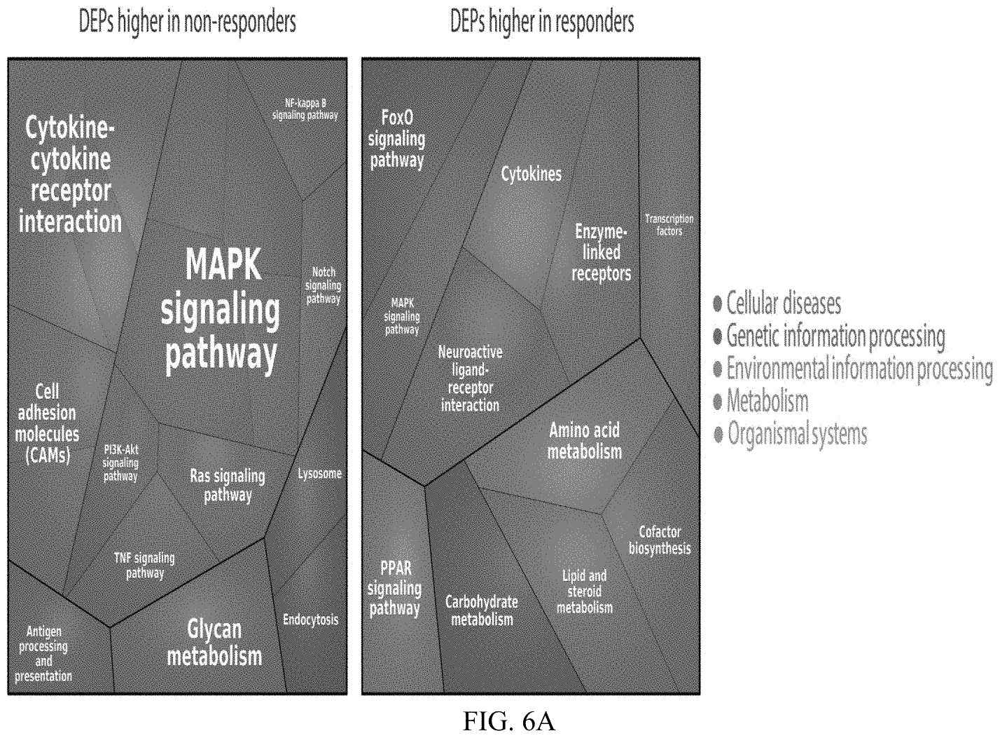

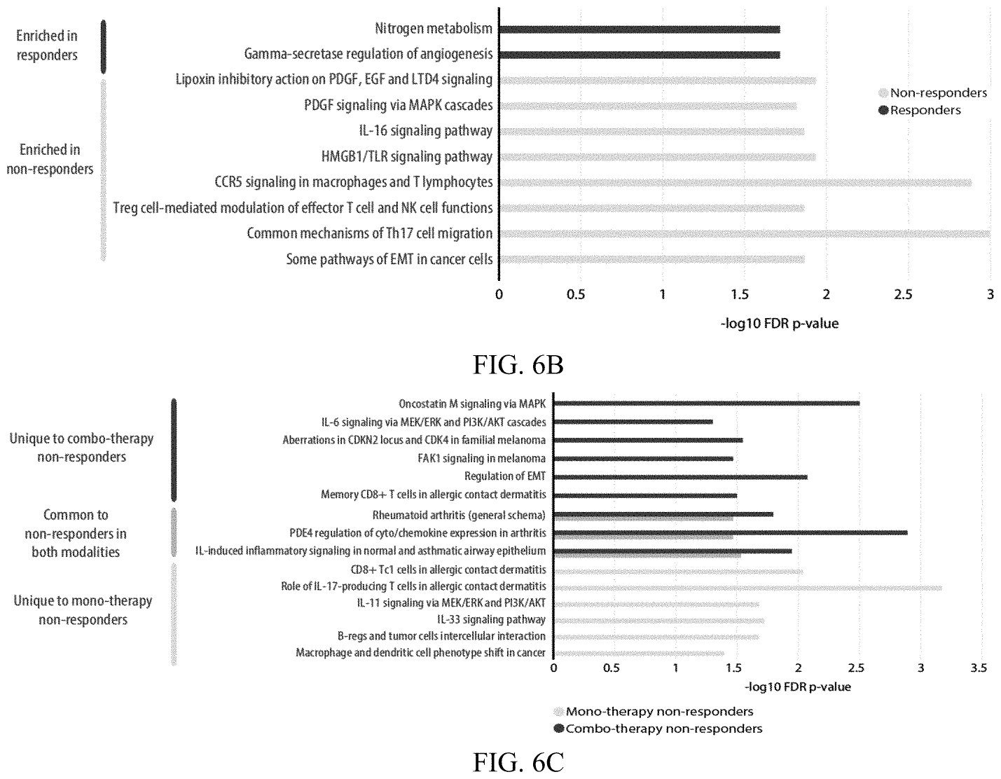

In order to get the strongest proteins with the highest potential to capture the biological differences between the two groups and the host-response effect of the therapy, the focus was on proteins that passed both one-sample t-test (which identifies proteins that change between T0 and T1) and two-sample t-test (which identifies proteins that differ between responders and non-responders). Using this approach, DEPs in the entire cohort and in two subsets of patients (two different treatment modalities-monotherapy of anti-PD1 or combination therapy of anti-PD1+anti-CTLA4) were identified. The DEPs of the entire cohort reveal differential functional groups, among them, differences in MAPK signaling pathway or in metabolism related proteins. FIG. 6A illustrates differentially expressed proteins (DEPs) identified in the plasma. The proteins are displayed using Proteomaps in a Voronoi plot where each polygon designate a DEP, and the size correlates with the T0 to T1 change. The DEPs are grouped into KEGG functional groups.

Next, enrichment analysis was performed using MetaCore with the DEPs as an input following multiple comparison correction using FDR adjusted p-value<0.05. The non-responder enrichment analysis of the entire cohort reveled multiple pathways associated with immune suppression, such as the involvement of T regulatory cells, as well as pathways that involve cancer progression or skin sensitization (FIG. 6B). The latter could be part of the side effects of the immunotherapy on the patients.

An additional enrichment analysis of the non-responder DEPs in each treatment modality (monotherapy vs. combination therapy) revealed processes that are unique to each modality. Some of the significantly enriched pathways involve immune suppression and may be part of the host-response to immunotherapy that attenuates response to the treatment (FIG. 6C).

### Example 2—Lung Cancer Cohort

A cohort comprised of 33 stage IV NSCLC patients treated with anti-PD1 (either Nivolumab or Pembrolizumab) was assembled; The response to treatment was determined either using response evaluation criteria in solid tumors (RECIST) 1.1 or estimated based on clinical evaluation. Out of the 33 samples, 15 patients were defined as responders (including: complete responders, CR; partial responders, PR; and stable disease, SD), and 18 were defined as non-responders (including progressive disease, PD). For each patient, plasma samples pre- (T0) and early on- (T₁) treatment were collected, and the proteomic changes following anti-PD1 treatment was determined. In total, 760 proteins were evaluated per sample. Following data normalization and quality control, checks were performed to identify technical biases and technical outliers (no outliers were removed in this analysis). Aiming to extract a predictive signature for response to treatment based on host-response related changes, the T₁/T₀ ratios (fold change) for each protein following log 2 transformation were examined. Next, proteins with values below limit of detection (LOD) were filtered out, leaving 418 proteins in total for the analysis.

The bioinformatic analysis was done in a multi-layer approach. Following quality check and normalization of the data, the analysis continued in two parallel tracks. One track directed for classification, aiming to generate a classifier that would enable prediction of response to treatment based on host-response data, as reflected by measuring the changes between T₀ and T₁. The second track aimed to identify driver proteins. It involves examination of the proteins in a functional group perspective, by applying advanced pathway enrichment tools. Further analysis by causal reasoning enabled identification of driver proteins that can initiate the enriched processes identified in the analysis.

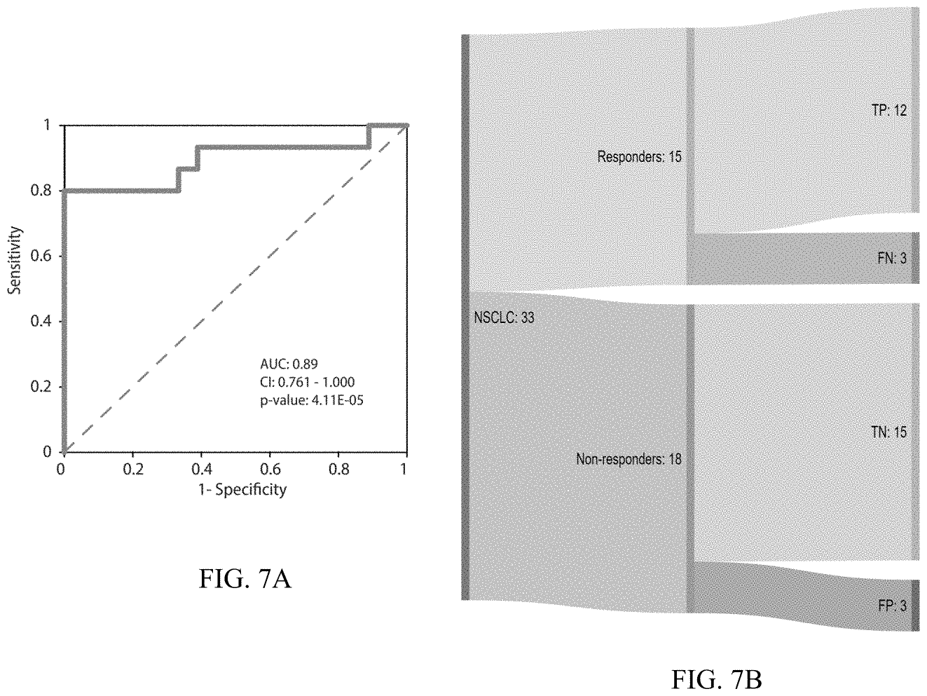

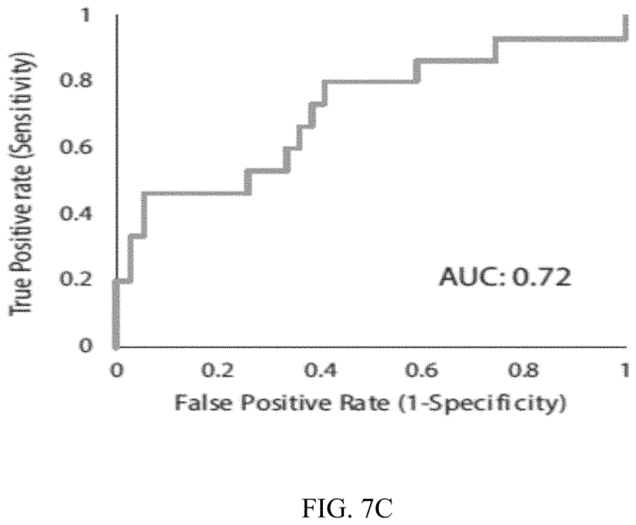

Next, Support Vector Machine (SVM) algorithm was used for discovering potential predictive signature for response based on host-response. Overall, a 3-protein signature was identified for the NSCLC indication receiving anti-PD1 treatment. The 3-protein signature had a high predictive power, as indicated by the area under the curve (AUC) of the receiver operating characteristics (ROC) plot of 0.89 (FIG. 7A). The results of the confusion matrix are indicated in a Sankey plot (FIG. 7B). The cut-off point on the predicted probabilities scale was set to identify responders with 93% sensitivity, resulting in specificity of 61%. The threshold of sensitivity was set to be above 90% in order to avoid classifying a responder as a non-responder.

To validate the results on an independent cohort, a validation cohort comprised of 54 samples from stage IIIB-IV NSCLC patients undergoing anti-PD1 treatment, of whom 15 were responders and 39 were non-responders, was assembled. The 3-protein signature was examined in a blind test, i.e. without indicating the response annotation for any of the samples. The AUC of the ROC curve of the validation set was 0.72, with a significant p-value of 0.013 (FIG. 7C). As in the training set performance analysis, we set the cutoff of high sensitivity (above 90%) to identify responders with 93% sensitivity, resulting in specificity of 26%.

### Enrichment Analysis

Next, we aimed to characterize the host response in the context of differentiating biological processes (DBPs) that change upon treatment and are different between responders and non-responders. This analysis is then used as the basis for the identification of proteins, termed herein “driver proteins”, that can be potential targets for intervention, such as part of a combination therapy with the specified therapy. To this end, the proteomic data may be analyzed using and proteomic tool, including but not limited to the Key Pathway Advisor (KPA) commercial tool from Clarivate Analytics.

Before performing the enrichment analysis that can identify the DBPs, a statistical test that selects the DEPs, whose levels change between T0 and T1 in responders and non-responders, was applied. The DEPs serve as an input for the enrichment analysis. To this end, a separate one-sample student's t-test was performed on each group. Overall, 42 and 40 DEPs were identified in the non-responder and responder groups, respectively (p-value<0.05).

Using the selected DEPS as an input, an enrichment analysis was performed using KPA under default settings of p-value thresholds of 0.05 and 0.01 for the enrichment analysis and the causal reasoning, respectively. Overall, there were 112 significantly enriched pathways in the non-responders and one enriched pathway in the responders. Out of the 112 non-responder enriched pathways, 21 pathways had a prediction for the direction of change based on causal reasoning (upregulated or downregulated in T1 compared with T0). Among the differentiating biological pathways, one can find multiple pathways related to immune response, involving either immune cell differentiation, or interleukin associated signaling. In addition, there are multiple processes associated with cell adhesion and extracellular matrix (ECM) regulation. In the responder group, on the other hand, only a single pathway was enriched.

Using KPA causal reasoning it is possible to identify the driver proteins potentially involved in the host-response related DBPs. In the non-responders, 979 driver proteins were identified, while in the responder group 5 driver proteins were identified.

### Example 3—Prediction Response in Psoriasis Afflicted Patients

A classifier based on the algorithm presented herein was trained to predict response to treatment in psoriasis patients. The data used for this analysis was taken from Lewis E. Tomalin et al. Early Quantification of Systemic Inflammatory Proteins Predicts Long-Term Treatment Response to Tofacitinib and Etanercept, Journal of Investigative Dermatology (2020) 140, 1026-1034, in which blood samples were taken from 140 patients (96 responders and 44 non-responders) with moderate-to-severe chronic plaque psoriasis, treated with the j anus kinase inhibiting compound tofacitinib (Xeljanz, 10 mg twice per day). A total of 92 inflammation associated (INF) proteins, as well as 65 proteins associated with cardiovascular disease (CVD), were determined in the blood samples that were taken immediately before treatment (WO, baseline) and 4 weeks posttreatment (W4). Response to treatment was based on PASI75 (Psoriasis Area Severity Index [PASI] 75 at week-12), which is the classical efficacy endpoint for psoriasis, where a patient is deemed a responder if the PASI decreases by >75% after 12 weeks of treatment and otherwise is a non-responder. A classifier for predicting whether a given patient will be a PASI-75 responder after 12-weeks of treatment was produced using the method described herein.

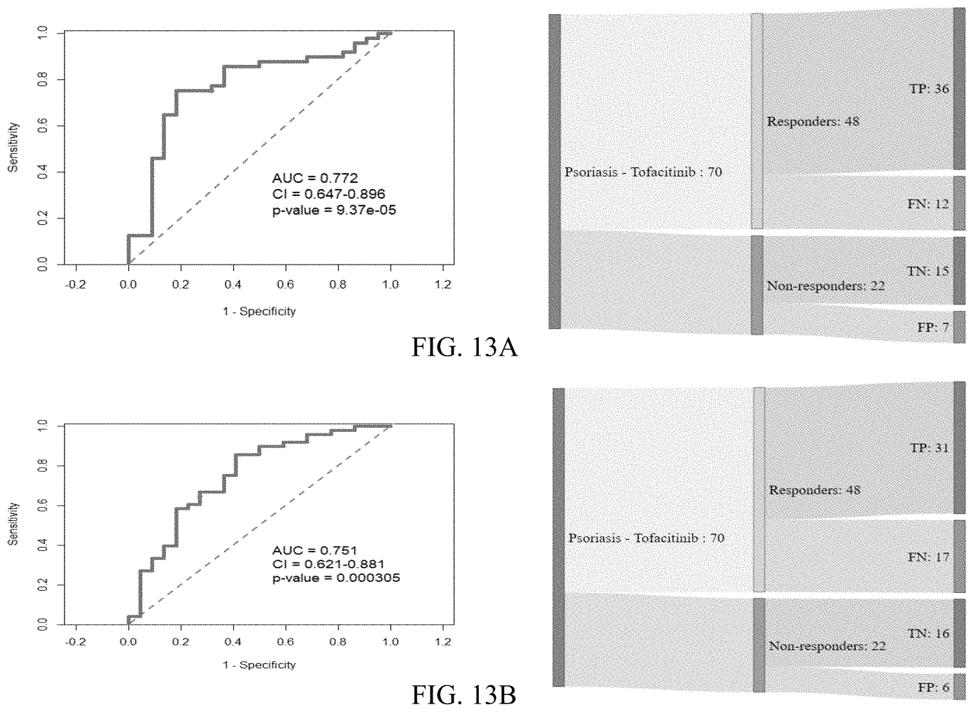

For this purpose, the data derived from the Tofacitinib treatment of psoriasis was randomly divided into two subsets of equal size. The first was used for training the machine learning algorithm, and the second for validation of the algorithm results. A predictive signature was identified (FIG. 13A, AUC ROC=0.772), that passed validation (FIG. 13B, AUC ROC=0.751). This predictive signature was limited to three protein features to minimize the probability of overfitting. This signature combines week4 (T1) and fold-change data showing that the system and method of the invention may be applied to determine responsiveness of psoriasis (i.e., and additional conditions other than cancer).

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Rather, the computer readable storage medium is a non-transient (i.e., not-volatile) medium.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language, R, Python or other programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The description of a numerical range should be considered to have specifically disclosed all the possible subranges as well as individual numerical values within that range. For example, description of a range from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A system for predicting a response in a target patient to a specified therapy, comprising:
at least one hardware processor; and a non-transitory computer-readable storage medium having stored thereon program instructions, the program instructions executable by the at least one hardware processor to:
receive, for each of a plurality of subjects having a specified type of disease and receiving a specified therapy for treating said disease, (a) a first biological signature associated with a biological sample collected at a first time point relative to said specified therapy, and (b) a second biological signature associated with a biological sample collected at a second time point relative to said specified therapy, wherein each one of said first and second biological signatures is selected from a list consisting of: a DNA profile, an RNA profile, a protein profile, a metabolomics profile, microbiome profile, a genomics profile, a transcriptomics profile, a cellular profile, an epigenomics profile, a post-translational modification-based profile, cellular profile, single-cell based analysis and a regulatory RNA profile,
calculate, for each of said plurality of subjects, a set of values representing a relation between said first and second biological signatures associated with said respective subject, and
at a training stage, train a machine learning model on a training set comprising:
(i) said calculated sets of values, and
(ii) labels associated with an outcome of said specified therapy in each of said subjects, to generate a classifier suitable for predicting a response in a target patient to said specified therapy, wherein said sets of values are labeled with said labels.

2. (canceled)

3. The system of claim 1, wherein said first and second biological signatures are each protein expression profiles, and said sets of values each comprise, with respect to each protein in said protein expression profiles, a ratio or a difference in the levels of expression of said protein in said first and second biological signatures, wherein said protein expression profile comprises expression values for at least two proteins.

4. (canceled)

5. The system of any one of claim 1, wherein said program instructions are further executable to perform a dimensionality reduction stage with respect to said sets of values, to reduce the number of variables in each of said sets of values, and wherein said dimensionality reduction stage identifies a subset of principal proteins in each of said sets of values.

6. (canceled)

7. The system of claim 5, wherein said training set comprises only said subset of principal proteins in each of said sets of values.

8. (canceled)

9. The system of claim 1, wherein each of said biological samples is one of: blood plasma, whole blood, blood serum, cerebrospinal fluid (CSF), and peripheral blood mononuclear cells (PBMCs).

10. The system of claim 1, wherein said specified type of disease is cancer.

11. (canceled)

12. (canceled)

13. (canceled)

14. The system of claim 1, wherein said predicting comprises an indication of secondary effects in said target subject.

15. The system of claim 1, wherein said program instructions are further executable to determine, based, at least in part, on said predicting, at least one of: continuing said specified therapy in said target subject, adjusting said specified therapy in said target subject, discontinuing said specified therapy in said target subject, and administering a different therapy to said target subject.

16. The system of claim 1, wherein said specified therapy is an immunotherapy, wherein said immunotherapy is selected from anti-PD-1/PD-L1 therapy, anti-CTLA-4 therapy, and both.

17. (canceled)

18. A method of predicting a response in a target patient to a specified therapy, comprising:
receiving, for each of a plurality of subjects having a specified type of disease and receiving a specified therapy for treating said disease, (a) a first biological signature associated with a biological sample collected at a first time point relative to said specified therapy, and (b) a second biological signature associated with a biological sample collected at a second time point relative to the specified therapy, wherein each one of said first and second biological signatures is selected from a list consisting of: a DNA profile, an RNA profile, a protein profile, a metabolomics profile, microbiome profile, a genomics profile, a transcriptomics profile, a cellular profile, an epigenomics profile, a post-translational modification-based profile, cellular profile, single-cell based analysis and a regulatory RNA profile;
calculating, for each of said plurality of subjects, a set of values representing a relation between said first and second biological signatures associated with said respective subject; and
at a training stage, training a machine learning model on a training set comprising:
(i) said calculated sets of values, and
(ii) labels associated with an outcome of said specified therapy in each of said subjects;
thereby generating a classifier suitable for predicting a response in said target patient to said specified therapy, wherein said sets of values are labeled with said labels.

19. (canceled)

20. The method of claim 18, wherein said first and second biological signatures are each protein expression profiles, and said sets of values each comprise, with respect to each protein in said protein expression profiles, a ratio of levels of expression of said protein in said first and second biological signatures, wherein said protein expression profile comprises expression values for at least two proteins.

21. (canceled)

22. The method of claim 18, further comprising performing a dimensionality reduction stage with respect to said sets of values, to reduce the number of variables in each of said sets of values, wherein said dimensionality reduction stage identifies a subset of principal proteins in each of said sets of values.

23. (canceled)

24. The method of claim 22, wherein said training set comprises only said subset of principal proteins in each of said sets of values.

25. (canceled)

26. The method of claim 18, wherein each of said biological samples is one of: blood plasma, whole blood, blood serum, cerebrospinal fluid (CSF), and peripheral blood mononuclear cells (PBMCs).

27. The method of claim 18, wherein said specified type of disease is a proliferative disease.

28. (canceled)

29. The method of claim 18, wherein said training set further comprises, with respect to at least some of said subjects, labels associated with clinical data.

30. (canceled)

31. The method of claim 18, further comprising at an inference stage, applying said classifier to a target set of said values associated with a target subject, thereby predicting a response in said target subject to said specified therapy.

32. The method of claim 31, further comprising determining, based, at least in part, on said predicting, at least one of: continuing said specified therapy in said target subject, adjusting said specified therapy in said target subject, discontinuing said specified therapy in said target subject, and administering a different therapy to said target subject.

33. (canceled)

34. The method of claim 32, wherein adjusting said specified therapy or administering a different therapy to said target subject is determined by a method comprising:
determining differentially expressed proteins (DEPs) between responders and non-responders;
determining, in the sample obtained from said subject, one or more resistance associated proteins (RAPs), selected from the DEPs; and
identifying a therapy suitable for balancing the level of the one or more RAPs in said subject.

35. The method of claim 32 wherein determining the one or more RAPs is by providing a probabilistic measurement of a distance of the DEP expression level from a defined group of samples selected from the responder group or the non-responder group, and wherein determining the one or more RAPs is by determining the expression distribution of each DEP in each of the responder and non-responder groups, fitting a probability density function for each group, and calculating for each subject, and based on the DEP expression of said subject, the probability of the DEP to be associated with one of the response groups.

36. (canceled)

37. (canceled)
